(19) United States
(12) Patent Application Publication
(10) Pub. No.: US 2005/0250713 A1
(43) Pub. Date: Nov. 10, 2005

(54) 11,12-Cyclic thiocarbamate macrolide antibacterial agents

(76) Inventors: Bin Zhu, Hillsborough, NJ (US); Brett Marinelli, Hamilton, NJ (US); Mark J. Macielag, Branchburg, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/114,502

(22) Filed: Apr. 26, 2005

Related U.S. Application Data
(60) Provisional application No. 60/565,447, filed on Apr. 26, 2004.

(51) Int. Cl. A61K 31/7048; C07H 17/08

(52) U.S. Cl. 514/29; 536/7.4

(57) ABSTRACT

The present invention is directed to novel macrolide antibacterial agents and processes for preparing them. The present invention is further directed to pharmaceutical compositions containing the novel macrolide antibacterial agents disclosed herein and their use in the treatment and prevention of bacterial infections.

### FIELD OF THE INVENTION

The present invention relates to the field of macrolide compounds having antibacterial activity, pharmaceutical compositions containing the compounds, and methods of treating bacterial infections with the compounds.

### BACKGROUND OF THE INVENTION

Erythromycins are well-known antibacterial agents widely used to treat and prevent bacterial infection caused by Gram-positive and Gram-negative bacteria. However, due to their low stability in acidic environment, they often carry side effects such as poor and erratic oral absorption. As with other antibacterial agents, bacterial strains having resistance or insufficient susceptibility to erythromycin have developed over time and are identified in patients suffering from such ailments as community-acquired pneumonia, upper and lower respiratory tract infections, skin and soft tissue infections, meningitis, hospital-acquired lung infections, and bone and joint infections. Particularly problematic pathogens include methicillin-resistant Staphylococcus aureus (MRSA), vancomycin-resistant enterococci (VRE) and penicillin- and macrolide-resistant Streptococcus pneumoniae. Therefore, continuing efforts are called for to identify new erythromycin derivative compounds with improved antibacterial activity, and/or unanticipated selectivity against various target microorganisms, particularly erythromycin-resistant strains.

The following references relate to various erythromycin derivatives disclosed as having antibacterial activity:

EP 216,169 and U.S. Pat. No. 4,826,820 to Brain et al. disclose antibacterially active 6-carbamate erythromycin derivatives stated to “have antibacterial properties, in particular against Gram-positive bacteria but also against some Gram-negative bacteria.”

U.S. Pat. No. 5,444,051, U.S. Pat. No. 5,561,118, and U.S. Pat. No. 5,770,579, all to Agouridas et al., disclose erythromycin compounds such as those of the formulae 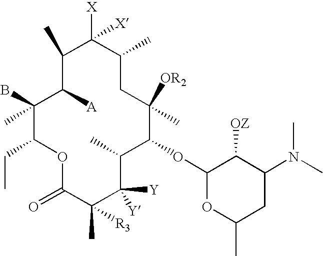
wherein substituents are as described in the respective references, which are all stated to be useful as antibiotics.

WO 97/17356 (Or et al.) discloses tricyclic erythromycin derivatives stated to be useful in the treatment and prevention of bacterial infections.

U.S. Pat. No. 5,866,549 to Or et al. and WO 98/09978 (Or et al.) disclose 6-O-substituted ketolides stated to have increased acid stability relative to erythromycin A and 6-O-methyl erythromycin A and enhanced activity toward gram negative bacteria and macrolide resistant gram positive bacteria.

WO 99/21871 (Phan et al.) discloses 2-halo-6-O-substituted ketolide derivatives of the formula 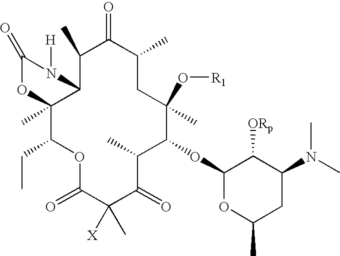
wherein substituents are as described in the respective reference, which are stated to possess antibacterial activity.

U.S. Pat. No. 6,169,168 to Asaka et al. discloses erythromycin A derivatives stated to “have a strong antibacterial activity not only against sensitive bacteria but also resistant bacteria.”

WO 98/23628 (Asaka et al.) discloses erythromycin A derivatives stated to have “a potent antibacterial activity against not only conventional erythromycin-sensitive bacteria but also erythromycin-resistant bacteria.”

WO 99/11651 (Or et al.) discloses 3-descladinose 6-O-substituded erythromycin derivatives for treating bacterial infections.

WO 99/21869 and WO 99/21870 (both to Asaka et al.) discloses erythromycin A derivatives stated to have “a strong antibacterial activity against not only erythromycin-sensitive bacteria but also erythromycin-resistant bacteria.”

WO 00/12522 (Randolph et al.) discloses 3′-N-desmethyl-3′-N-substituted-6-O-methyl-11,12-cyclic carbamate erythrolide A derivatives as antagonists of luteinizing hormone-releasing hormone.

WO 00/26224 (Kaneko) discloses novel macrolide antibiotics useful as potent antibacterial and antiprotozoal agents.

WO 00/75156 (Phan et al.) discloses 6-O-carbamate ketolide compounds stated to be useful for treatment and prevention of infections in a mammal.

EP 1146051 (Kaneko et al.) discloses erythromycin A and ketolide derivatives useful for the treatment of a bacterial or protozoal infection in a mammal.

EP 1114826 (Kaneko et al.) discloses erythromycin macrolide derivatives as antibacterial and prokinetic agents.

WO 00/71557 to Dirlam et al. discloses 13-methylerythromycin derivatives that are useful as antibacterial and antiprotozoal agents in mammals (including humans), fish and birds.

WO 01/10878 (Asaka et al.) discloses erythromycin derivatives stated to be “characterized by an acyl group introduced at the 3-position, a cyclic carbamate structure fused at the 11- and 12-positions, and a five-membered heterocycle on the 11-position substituent, one of the nitrogen atoms of which is bonded to the 11-position nitrogen atom through an alkyl group.”

WO 02/26753 (Kato et al.) discloses erythromycin A derivatives as antimicrobial agents.

U.S. Pat. No. 5,922,683 to Or et al. discloses multicyclic erythromycin compounds having antibacterial activity.

U.S. Pat. No. 6,355,620 to Ma et al. discloses C-2 modified erythromycin derivatives that are useful in treating bacterial infections.

WO 02/46204 and US 2002115620 (both to Henninger et al.) disclose 6-O-carbamoyl ketolide derivatives of erythromycin useful as antibacterials.

WO 03004509 (Chu et al.) discloses C12 modified erythromycin macrolides and ketolides useful in the treatment of bacterial infections.

WO 03/024986 to Grant et al. discloses 6-O-carbamate-11,12-lactoketolides with antibacterial activity.

WO 03/050132 to Henninger et al. discloses 6-O-acylketolide derivatives of erythromycin useful as antibacterials.

WO 03/093289 to Gu et al. describes tricyclic macrolide erythromycin derivatives with antibacterial activity.

WO 03/090760 to Ma et al. describes macrolide oxolide erythromycin derivatives useful for prophylaxis or treatment of bacterial infections.

### SUMMARY OF THE INVENTION

The invention provides compounds of Formula 1: 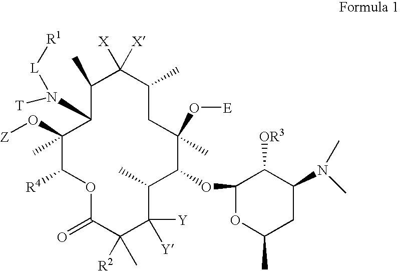
R¹ is selected from the group consisting of hydrogen, optionally substituted alkyl, optionally substituted alkenyl, optionally substituted alkynyl, cycloalkyl, optionally substituted aryl, and optionally substituted heteroaryl;
R² is selected from the group consisting of hydrogen, halogen, and hydroxy;
R³ is hydrogen or a hydroxy protecting group;
R⁴ is selected from the group consisting of hydrogen, C₁-C₁₀-alkyl, C₂-C₁₀-alkenyl, C₂-C₁₀-alkynyl, aryl, heteroaryl, heterocyclo, aryl(C₁-C₁₀)alkyl, aryl(C₂-C₁₀)alkenyl, aryl(C₂-C₁₀)alkynyl, heterocyclo(C₁-C₁₀)alkyl, heterocyclo(C₂-C₁₀)alkenyl, and heterocyclo(C₂-C₁₀)alkynyl, C₃-C₆-cycloalkyl, C₅-C₈-cycloalkenyl, alkoxyalkyl containing 1-6 carbon atoms in each alkyl or alkoxy group, and alkylthioalkyl containing 1-6 carbon atoms in each alkyl or thioalkyl group;
L is absent or C(O);
T is hydrogen;
Z is hydrogen, or T and Z may be taken together to form a thiocarbonyl group;
E is selected from the group consisting of optionally substituted C₁-C8-alkyl, optionally substituted C₃-C₈-alkenyl, and optionally substituted C₃-C8-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein
   Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 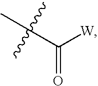
      wherein
W is selected from the group consisting of
(1) a substituted pyrrole of the formula 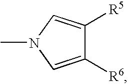
   wherein
R⁵ and R⁶ are independently selected from the group consisting of hydrogen, CN, nitro, —C(O)R⁷, —C(O)OR⁷, —C(O)NR⁷R⁸, —SO₂R⁷, C₁-C₈-alkyl, C₂-C₈-alkenyl, C₂-C8-alkynyl, C₃-C8-cycloalkyl, C₅-C8-cycloalkenyl, aryl, and heteroaryl, wherein
   R⁷ and R⁸ are independently selected from the group consisting of hydrogen, alkyl, alkenyl, alkynyl, cycloalkyl, aryl, heteroaryl, heterocyclo, aralkyl, heteroaralkyl, and heterocycloalkyl;
(2) NHR⁹, wherein
   R⁹ is independently selected from the group consisting of hydrogen, optionally substituted C₁-C8-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl; and
(3) OR⁹, wherein
   R⁹ is independently selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl;
   X and X′, together with the carbon atom to which they are attached, form C═O, C═NR_{c}, or C═NOR_{c}, wherein R_{c} is independently selected from the group consisting of hydrogen, alkyl, alkenyl and alkynyl; and
   Y and Y′, together with the carbon atom to which they are attached, form C═O, —CHOH, C═NR_{c}, or C═NOR_{c}, wherein R_{c} is independently selected from the group consisting of hydrogen, alkyl, alkenyl and alkynyl;with the following provisos:
      1) when L is absent and T and Z combine to form a thiocarbonyl group, R¹ is hydrogen;
      2) when E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, T and Z are both hydrogen;
      3) when T and Z are both hydrogen, E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b},
         wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 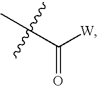
            wherein
W is NHR⁹, wherein
   R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl;
or an optical isomer, enantiomer, diastereomer, racemate or racemic mixture thereof, or a pharmaceutically acceptable salt, esters or pro-drugs thereof.

Compounds of the above formula are useful as antibacterial agents for the treatment of bacterial infections in a subject, such as a human or an animal.

The present invention is also directed to a method of treating a subject having a condition caused by or contributed to by bacterial infection, which comprises administering to the subject a therapeutically effective amount of the compound of Formula 1.

The present invention is further directed to a method of preventing a subject from suffering from a condition caused by or contributed to by bacterial infection, which comprises administering to the subject a prophylactically effective amount of the compound of Formula 1.

Other objects and advantages will become apparent to those skilled in the art from a review of the ensuing specification.

### DETAILED DESCRIPTION OF THE INVENTION

The invention provides compounds of Formula 1 useful as antibacterial agents for the treatment of bacterial infections in a subject, such as a human or animal: 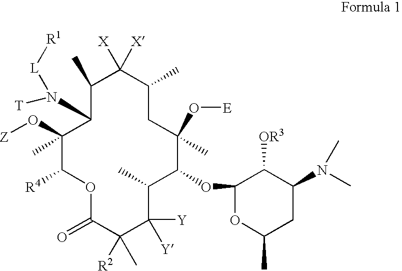
R¹ is selected from the group consisting of hydrogen, optionally substituted alkyl, optionally substituted alkenyl, optionally substituted alkynyl, cycloalkyl, optionally substituted aryl, and optionally substituted heteroaryl;
R² is selected from the group consisting of hydrogen, halogen, and hydroxy;
R³ is hydrogen or a hydroxy protecting group;
R⁴ is selected from the group consisting of hydrogen, C₁-C₁₀-alkyl, C₂-C₁₀-alkenyl, C₂-C₁₀-alkynyl, aryl, heteroaryl, heterocyclo, aryl(C₁-C₁₀)alkyl, aryl(C₂-C₁₀)alkenyl, aryl(C₂-C₁₀)alkynyl, heterocyclo(C₁-C₁₀)alkyl, heterocyclo(C₂-C₁₀)alkenyl, and heterocyclo(C₂-C₁₀)alkynyl, C₃-C₆-cycloalkyl, C₅-C₈-cycloalkenyl, alkoxyalkyl containing 1-6 carbon atoms in each alkyl or alkoxy group, and alkylthioalkyl containing 1-6 carbon atoms in each alkyl or thioalkyl group;
L is absent or C(O);
T is hydrogen;
Z is hydrogen, or T and Z may be taken together to form a thiocarbonyl group;
E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein
   Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 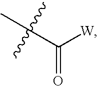
      wherein
W is selected from the group consisting of
(1) a substituted pyrrole of the formula 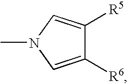
   wherein
R⁵ and R⁶ are independently selected from the group consisting of hydrogen, CN, nitro, —C(O)R⁷, —C(O)OR⁷, —C(O)NR⁷R⁸, —SO₂R⁷, C₁-C₈-alkyl, C₂-C₈-alkenyl, C₂-C₈-alkynyl, C₃-C₈-cycloalkyl, C₅-C₈-cycloalkenyl, aryl, and heteroaryl, wherein
   R⁷ and R⁸ are independently selected from the group consisting of hydrogen, alkyl, alkenyl, alkynyl, cycloalkyl, aryl, heteroaryl, heterocyclo, aralkyl, heteroaralkyl, and heterocycloalkyl;
(2) NHR⁹, wherein
   R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl; and
(3) OR⁹, wherein
   R⁹ is independently selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl;
   X and X′, together with the carbon atom to which they are attached, form C═O, C═NR_{c}, or C═NOR_{c}, wherein R_{c} is independently selected from the group consisting of hydrogen, alkyl, alkenyl and alkynyl; and
   Y and Y′, together with the carbon atom to which they are attached, form C═O, —CHOH, C═NR_{c}, or C═NOR_{c}, wherein R_{c} is independently selected from the group consisting of hydrogen, alkyl, alkenyl and alkynyl;with the following provisos:
      1) when L is absent and T and Z combine to form a thiocarbonyl group, R¹ is hydrogen;
      2) when E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, T and Z are both hydrogen;
      3) when T and Z are both hydrogen, E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein
         Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 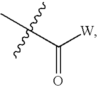
            wherein
W is NHR⁹, wherein
   R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl;
or an optical isomer, enantiomer, diastereomer, racemate or racemic mixture thereof, or a pharmaceutically acceptable salt, esters or pro-drugs thereof.

Relative to the above description, certain definitions apply as follows.

Unless otherwise noted, under standard nomenclature used throughout this disclosure the terminal portion of the designated side chain is described first, followed by the adjacent functionality toward the point of attachment.

Unless specified otherwise, the terms “alkyl”, “alkenyl”, and “alkynyl,” whether used alone or as part of a substituent group, include straight and branched chains having 1 to 8 carbon atoms, or any number within this range. The term “alkyl” refers to straight or branched chain hydrocarbons. “Alkenyl” refers to a straight or branched chain hydrocarbon with at least one carbon-carbon double bond. “Alkynyl” refers to a straight or branched chain hydrocarbon with at least one carbon-carbon triple bound. For example, alkyl radicals include methyl, ethyl, n-propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. “Alkoxy” radicals are oxygen ethers formed from the previously described straight or branched chain alkyl groups. “Cycloalkyl” groups contain 3 to 8 ring carbons and preferably 5 to 7 ring carbons. The alkyl, alkenyl, alkynyl, cycloalkyl group and alkoxy group may be independently substituted with one or more members of the group including, but not limited to, halogen, alkyl, alkenyl, alkynyl, cycloalkyl, oxo, aryl, heteroaryl, heterocyclo, CN, nitro, —OCORₐ, —ORₐ, —SRₐ, —SORₐ, —SO₂Rₐ, —COORₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from H, alkyl, alkenyl, alkynyl, cycloalkyl, aryl, heteroaryl, heterocyclo, aralkyl, heteroaralkyl, and heterocycloalkyl.

The term “acyl” as used herein, whether used alone or as part of a substituent group, means an organic radical having 2 to 6 carbon atoms (branched or straight chain) derived from an organic acid by removal of the hydroxyl group. The term “Ac” as used herein, whether used alone or as part of a substituent group, means acetyl.

The term “halo” or “halogen” means fluoro, chloro, bromo and iodo. (Mono-, di-, tri-, and per-)haloalkyl is an alkyl radical substituted by independent replacement of the hydrogen atoms thereon with halogen.

“Aryl” or “Ar,” whether used alone or as part of a substituent group, is a carbocyclic aromatic radical including, but not limited to, phenyl, 1- or 2-naphthyl and the like. The carbocyclic aromatic radical may be substituted by independent replacement of 1 to 3 of the hydrogen atoms thereon with halogen, OH, CN, mercapto, nitro, amino, C₁-C₈-alkyl, C₁-C₈-alkoxyl, C₁-C₈-alkylthio, C₁-C₈-alkylamino, di(C₁-C₈-alkyl)amino, (mono-, di-, tri-, and per-)haloalkyl, formyl, carboxy, alkoxycarbonyl, C₁-C₈-alkyl-CO—O—, C₁-C₈-alkyl-CO—NH—, or carboxamide. Illustrative aryl radicals include, for example, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimethylcarbamylphenyl and the like. “Ph” or “PH” denotes phenyl.

Whether used alone or as part of a substituent group, “heteroaryl” refers to a cyclic, fully unsaturated radical having from five to ten ring atoms of which one ring atom is selected from S, O, and N; 0-2 ring atoms are additional heteroatoms independently selected from S, O, and N; and the remaining ring atoms are carbon. The radical may be joined to the rest of the molecule via any of the ring atoms. Exemplary heteroaryl groups include, for example, pyridinyl, pyrazinyl, pyrimidinyl, pyridazinyl, pyrrolyl, pyrazolyl, imidazolyl, thiazolyl, oxazolyl, isoxazolyl, thiadiazolyl, triazolyl, triazinyl, oxadiazolyl, thienyl, furanyl, quinolinyl, isoquinolinyl, indolyl, isothiazolyl, N-oxo-pyridyl, 1,1-dioxothienyl, benzothiazolyl, benzoxazolyl, benzothienyl, quinolinyl-N-oxide, benzimidazolyl, benzopyranyl, benzisothiazolyl, benzisoxazolyl, benzodiazinyl, benzofurazanyl, indazolyl, indolizinyl, benzofuryl, cinnolinyl, quinoxalinyl, indazolyl, pyrrolopyridinyl, furopyridinyl (such as furo[2,3-c]pyridinyl, furo[3,2-b]pyridinyl, or furo[2,3-b]pyridinyl), imidazopyridinyl (such as imidazo[4,5-b]pyridinyl or imidazo[4,5-c]pyridinyl), naphthyridinyl, phthalazinyl, purinyl, pyridopyridyl, quinazolinyl, thienofuryl, thienopyridyl, and thienothienyl. The heteroaryl group may be substituted by independent replacement of 1 to 3 of the hydrogen atoms thereon with halogen, OH, CN, mercapto, nitro, amino, C₁-C₈-alkyl, C₁-C₈-alkoxyl, C₁-C₈-alkylthio, C₁-C₈-alkylamino, di(C₁-C₈-alkyl)amino, (mono-, di-, tri-, and per-)haloalkyl, formyl, carboxy, alkoxycarbonyl, C₁-C₈-alkyl-CO—O—, C₁-C₈-alkyl-CO—NH—, or carboxamide. Heteroaryl may be substituted with a mono-oxo to give for example a 4-oxo-1H-quinoline.

The terms “heterocycle,” “heterocyclic,” and “heterocyclo” refer to an optionally substituted, fully saturated, partially saturated, or non-aromatic cyclic group which is, for example, a 4- to 7-membered monocyclic, 7- to 11-membered bicyclic, or 10- to 15-membered tricyclic ring system, which has at least one heteroatom in at least one carbon atom containing ring. Each ring of the heterocyclic group containing a heteroatom may have 1, 2, or 3 heteroatoms selected from nitrogen atoms, oxygen atoms, and sulfur atoms, where the nitrogen and sulfur heteroatoms may also optionally be oxidized. The nitrogen atoms may optionally be quaternized. The heterocyclic group may be attached at any heteroatom or carbon atom. The heterocyclic group may be substituted by independent replacement of 1 to 3 of the hydrogen atoms thereon with aryl, heteroaryl, halogen, C₁-C₈-alkyl, C₁-C₈-alkoxyl, carboxy, alkoxycarbonyl, or carboxamide.

Exemplary monocyclic heterocyclic groups include pyrrolidinyl; oxetanyl; pyrazolinyl; imidazolinyl; imidazolidinyl; oxazolinyl; oxazolidinyl; isoxazolinyl; thiazolidinyl; isothiazolidinyl; tetrahydrofuryl; piperidinyl; piperazinyl; 2-oxopiperazinyl; 2-oxopiperidinyl; 2-oxopyrrolidinyl; 4-piperidonyl; tetrahydropyranyl; tetrahydrothiopyranyl; tetrahydrothiopyranyl sulfone; morpholinyl; thiomorpholinyl; thiomorpholinyl sulfoxide; thiomorpholinyl sulfone; 1,3-dioxolane; dioxanyl; thietanyl; thiiranyl; 2-oxazepinyl; azepinyl; and the like. Exemplary bicyclic heterocyclic groups include quinuclidinyl; tetrahydroisoquinolinyl; dihydroisoindolyl; dihydroquinazolinyl (such as 3,4-dihydro-4-oxo-quinazolinyl); dihydrobenzofuryl; dihydrobenzothienyl; benzothiopyranyl; dihydrobenzothiopyranyl; dihydrobenzothiopyranyl sulfone; benzopyranyl; dihydrobenzopyranyl; indolinyl; chromonyl; coumarinyl; isochromanyl; isoindolinyl; piperonyl; tetrahydroquinolinyl; and the like.

Substituted aryl, substituted heteroaryl, and substituted heterocycle may also be substituted with a second substituted-aryl, a second substituted-heteroaryl, or a second substituted-heterocycle to give, for example, a 4-pyrazol-1-yl-phenyl or 4-pyridin-2-yl-phenyl.

Designated numbers of carbon atoms (e.g., C₁₋₈) shall refer independently to the number of carbon atoms in an alkyl or cycloalkyl moiety or to the alkyl portion of a larger substituent in which alkyl appears as its prefix root.

Unless specified otherwise, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The term “hydroxy protecting group” refers to groups known in the art for such purpose. Commonly used hydroxy protecting groups are disclosed, for example, in T. H. Greene and P. G. M. Wuts, Protective Groups in Organic Synthesis, 2nd edition, John Wiley & Sons, New York (1991), which is incorporated herein by reference. Illustrative hydroxyl protecting groups include but are not limited to tetrahydropyranyl; benzyl; methylthiomethyl; ethythiomethyl; pivaloyl; phenylsulfonyl; triphenylmethyl; trisubstituted silyl such as trimethylsilyl, triethylsilyl, tributylsilyl, tri-isopropylsilyl, t-butyldimethylsilyl, tri-t-butylsilyl, methyldiphenylsilyl, ethyldiphenylsilyl, t-butyldiphenylsilyl; acyl and aroyl such as acetyl, pivaloylbenzoyl, 4-methoxybenzoyl, 4-nitrobenzoyl and phenylacetyl.

Where the compounds according to this invention have at least one stereogenic center, they may accordingly exist as enantiomers. Where the compounds possess two or more stereogenic centers, they may additionally exist as diastereomers. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Some of the compounds of the present invention may have trans and cis isomers. In addition, where the processes for the preparation of the compounds according to the invention give rise to mixture of stereoisomers, these isomers may be separated by conventional techniques such as preparative chromatography. The compounds may be prepared as a single stereoisomer or in racemic form as a mixture of some possible stereoisomers. The non-racemic forms may be obtained by either synthesis or resolution. The compounds may, for example, be resolved into their component enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography.

The phrase “a pharmaceutically acceptable salt” denotes one or more salts of the free base which possess the desired pharmacological activity of the free base and which are neither biologically nor otherwise undesirable. These salts may be derived from inorganic or organic acids. Examples of inorganic acids are hydrochloric acid, nitric acid, hydrobromic acid, sulfuric acid, or phosphoric acid. Examples of organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, salicyclic acid and the like. Suitable salts are furthermore those of inorganic or organic bases, such as KOH, NaOH, Ca(OH)₂, Al(OH)₃, piperidine, morpholine, ethylamine, triethylamine and the like.

Included within the scope of the invention are the hydrated forms of the compounds which contain various amounts of water, for instance, the hydrate, hemihydrate, and sesquihydrate forms. The present invention also includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The term “subject” includes, without limitation, any animal or artificially modified animal. As a particular embodiment, the subject is a human.

The term “drug-resistant” or “drug-resistance” refers to the characteristics of a microbe to survive in presence of a currently available antimicrobial agent such as an antibiotic at its routine, effective concentration.

The compounds described in the present invention possess antibacterial activity due to their novel structure, and are useful as antibacterial agents for the treatment of bacterial infections in humans and animals. In particular, compounds of the present invention have activity against Gram-positive and Gram-negative respiratory pathogens. The following are representative compounds of the present invention:

Compound of Formula 1 wherein R¹ is hydrogen, R² is hydrogen, R³ is hydrogen, R⁴ is ethyl, L is absent, X and X′, together with the carbon atom to which they are attached, form C═O, Y and Y′, together with the carbon atom to which they are attached, form C═O, T and Z are taken together to form a thiocarbonyl group, E is —C(O)—W, wherein W is (E)-NH—CH₂—CH═CH-[4-(2-pyrazinyl)phenyl];

Compound of Formula 1 wherein R¹ is hydrogen, R² is hydrogen, R³ is hydrogen, R⁴ is ethyl, L is absent, X and X′, together with the carbon atom to which they are attached, form C═O, Y and Y′, together with the carbon atom to which they are attached, form C═O, T and Z are taken together to form a thiocarbonyl group, E is —C(O)—W, wherein W is (E)-NH—CH₂—CH═CH-[4-(3-pyridazinyl)phenyl];

Compound of Formula 1 wherein R¹ is hydrogen, R² is hydrogen, R³ is hydrogen, R⁴ is ethyl, L is absent, X and X′, together with the carbon atom to which they are attached, form C═O, Y and Y′, together with the carbon atom to which they are attached, form C═O, T and Z are taken together to form a thiocarbonyl group, E is —C(O)—W, wherein W is (E)-NH—CH₂—CH═CH-(3-quinolinyl);

Compound of Formula 1 wherein R¹ is hydrogen, R² is hydrogen, R³ is hydrogen, R⁴ is ethyl, L is absent, X and X′, together with the carbon atom to which they are attached, form C═O, Y and Y′, together with the carbon atom to which they are attached, form C═O, T and Z are taken together to form a thiocarbonyl group, E is —C(O)—W, wherein W is (E)-NH—CH₂—CH═CH-[5-(2-bromopyridinyl)];

Compound of Formula 1 wherein R¹ is hydrogen, R² is hydrogen, R³ is hydrogen, R⁴ is ethyl, L is absent, X and X′, together with the carbon atom to which they are attached, form C═O, Y and Y′, together with the carbon atom to which they are attached, form C═O, T and Z are taken together to form a thiocarbonyl group, E is —C(O)—W, wherein W is (E)-NH—CH₂—CH═CH-[5-(2-cyclopropyl)pyrimidinyl];

Compound of Formula 1 wherein R¹ is hydrogen, R² is hydrogen, R³ is hydrogen, R⁴ is ethyl, L is absent, X and X′, together with the carbon atom to which they are attached, form C═O, Y and Y′, together with the carbon atom to which they are attached, form C═O, T and Z are taken together to form a thiocarbonyl group, E is —C(O)—W, wherein W is (E)-O—CH₂—CH═CH-(3-quinolinyl);

Compound of Formula 1 wherein R¹ is hydrogen, R² is hydrogen, R³ is hydrogen, R⁴ is ethyl, L is absent, X and X′, together with the carbon atom to which they are attached, form C═O, Y and Y′, together with the carbon atom to which they are attached, form C═O, T and Z are taken together to form a thiocarbonyl group, E is —C(O)—W, wherein W is (E)-O—CH₂—CH═CH-[5-(2-bromopyridinyl)];

Compound of Formula 1 wherein R¹ is hydrogen, R² is hydrogen, R³ is hydrogen, R⁴ is ethyl, L is absent, X and X′, together with the carbon atom to which they are attached, form C═O, Y and Y′, together with the carbon atom to which they are attached, form C═O, T and Z are taken together to form a thiocarbonyl group, E is —C(O)—W, wherein W is (Z)-NH—CH₂—CF═CH-[4-(2-pyrimidinyl)phenyl];

Compound of Formula 1 wherein R¹ is hydrogen, R² is hydrogen, R³ is hydrogen, R⁴ is ethyl, L is absent, X and X′, together with the carbon atom to which they are attached, form C═O, Y and Y′, together with the carbon atom to which they are attached, form C═O, T and Z are taken together to form a thiocarbonyl group, E is —C(O)—W, wherein W is (Z)-NH—CH₂—CF═CH-(3-quinolinyl).

Compound of Formula 1 wherein R¹ is 3-[4-(2-pyrimidinyl)phenyl]propyl, R² is hydrogen, R³ is hydrogen, R⁴ is ethyl, L is absent, X and X′, together with the carbon atom to which they are attached, form C═O, Y and Y′, together with the carbon atom to which they are attached, form C═O, T is hydrogen, Z is hydrogen, E is methyl.

This invention also provides processes for preparing the instant compounds. The compounds of Formula 1 may be prepared from readily available starting materials such as erythromycin and erythromycin derivatives well known in the art. Outlined in Schemes 1 through 15 are representative procedures to prepare the compounds of the instant invention:

Scheme 1 illustrates a method of synthesizing the 2′4″-diacetyl-6-carbamoyl-11,12-dideoxy-11,12-iminocarbonyloxyerythromycin A (VI) and the 2′-acetyl-6-carbamoyl-11,12-dideoxy-3-O-descladinosyl-11,12-iminocarbonyloxyerythromycin A (VII) precursors to the compounds of the invention.

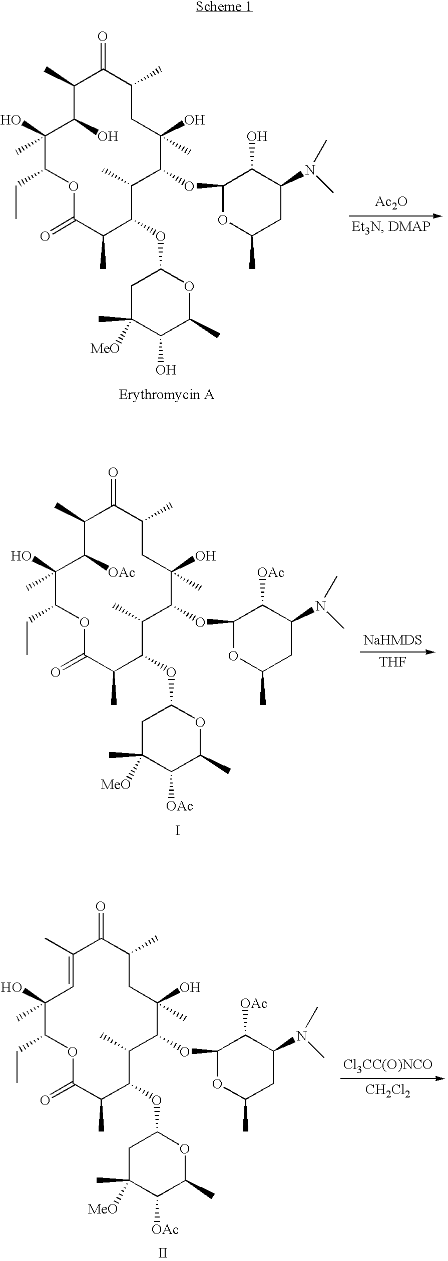

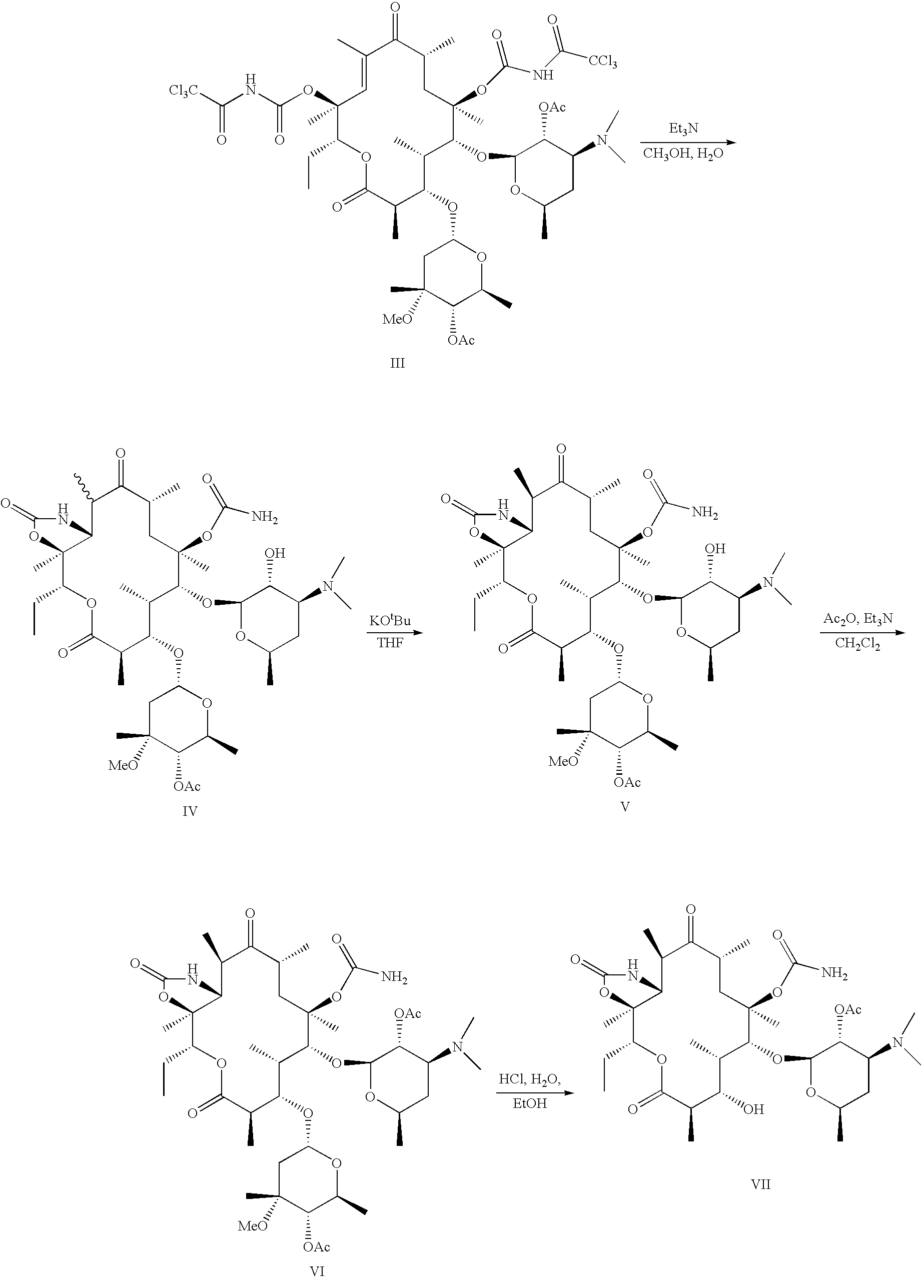

Erythromycin A is treated with acetic anhydride in the presence of a tertiary amine base, such as triethylamine, diisopropylethylamine, or pyridine, and an acylation catalyst, such as DMAP, in a suitable solvent such as methylene chloride, chloroform or THF at a temperature ranging from −20° C. to 37° C. for 2 to 48 hours to afford 2′,4″,11-triacetylerythromycin A (I). The 10,11-anhydro derivative (II) can be readily obtained by treatment of I with a base in an inert solvent such as THF, dioxane, DME, or DMF at a temperature ranging from −78° C. to 80° C. for 1-24 hours. Suitable bases to effect the elimination reaction include, but are not limited to, sodium hexamethyldisilazide, potassium hexamethyldisilazide, LDA, lithium tetramethylpiperidide, DBU, and tetramethylguanidine. It will be apparent to one skilled in the art that alternative methods for synthesis of 2′,4″-diacetyl-10,11-anhydroerythromycin A are available, including conversion of erythromycin A to the 11,12-cyclic carbonate derivative with ethylene carbonate, followed by elimination with tetramethylguanidine, as described in Hauske, J. R. and Kostek, G., J. Org. Chem. 1982, 47, 1595. Selective protection of the 2′ and 4″-hydroxyl groups can then be readily accomplished with acetic anhydride in the presence of a tertiary amine base. Likewise, alternative protecting group strategies may be employed. For example, erythromycin A may be treated with benzoic anhydride, propionic anhydride, or formic acetic anhydride under similar conditions as described above to obtain the 2′,4″,11-triacylated erythromycin A derivative followed by elimination to afford the corresponding 10,11-anhydro compound.

Once the suitably protected 10,11-anhydro derivative is obtained, derivatization of both tertiary hydroxyl groups can be carried out by treatment with trichloroacetylisocyanate in an inert solvent, such as methylene chloride, chloroform, or THF at a temperature ranging from −20° C. to 37° C. for 1-24 hours to yield the di-(N-trichloroacetyl)carbamate derivative (III). The N-trichloroacetylcarbamate functionalities can be hydrolyzed to the corresponding primary carbamates by treatment with a suitable base, such as triethylamine, in an aqueous solvent mixture, such as methanol/water for 1-24 hours at a temperature ranging from 20° C. to 80° C. Alternative bases may likewise be used to effect this conversion, such as sodium hydroxide, potassium hydroxide, sodium carbonate and potassium carbonate. Under the reaction conditions, the primary carbamate formed at the 12-position undergoes spontaneous Michael addition to the electrophilic 11-position of the □,□-unsaturated ketone and the 2′-acetoxy group is hydrolyzed to the corresponding hydroxyl to afford the cyclic carbamate derivative (IV). Compound IV is generally isolated as a mixture of methyl epimers at the C10-position, which can be readily converted to the desired C10-β-methyl epimer (V) by treatment with an equilibrating base, such as potassium t-butoxide, tetramethylguanidine, or DBU in a suitable solvent, such as THF, dioxane, DME, DMF or t-butanol at a temperature ranging from −78° C. to 80° C. for 1 to 24 hours. Reprotection of the 2′-hydroxyl group to give VI can be carried out by treatment with acetic anhydride in the presence of a tertiary amine base, such as triethylamine, diisopropylethylamine, or pyridine, and optionally an acylation catalyst, such as DMAP, in a suitable solvent such as methylene chloride, chloroform or THF at a temperature ranging from −20° C. to 37° C. for 2 to 48 hours. It is understood that an orthogonal protection strategy of the sugar hydroxyls may also be employed by treatment of V with alternate reagents such as benzoic anhydride, benzyl chloroformate, hexamethyldisilazane, or a trialkylsilyl chloride. Finally, selective removal of the cladinose sugar can be accomplished by reaction of VI with an acid, such as hydrochloric, sulfuric, chloroacetic, and trifluoroacetic, in the presence of alcohol and water to afford VII. Reaction time is typically 0.5-24 hours at a temperature ranging from −10° C. to 37° C.

Scheme 2 depicts the synthesis of compounds of formulae IX, X, XI, XII, XIII, 1a, and 1b.

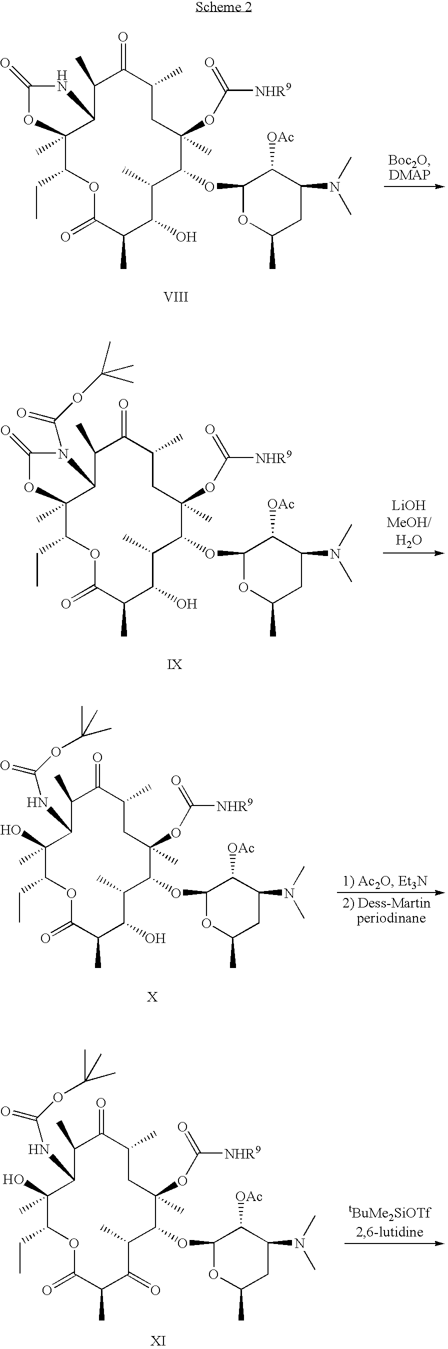

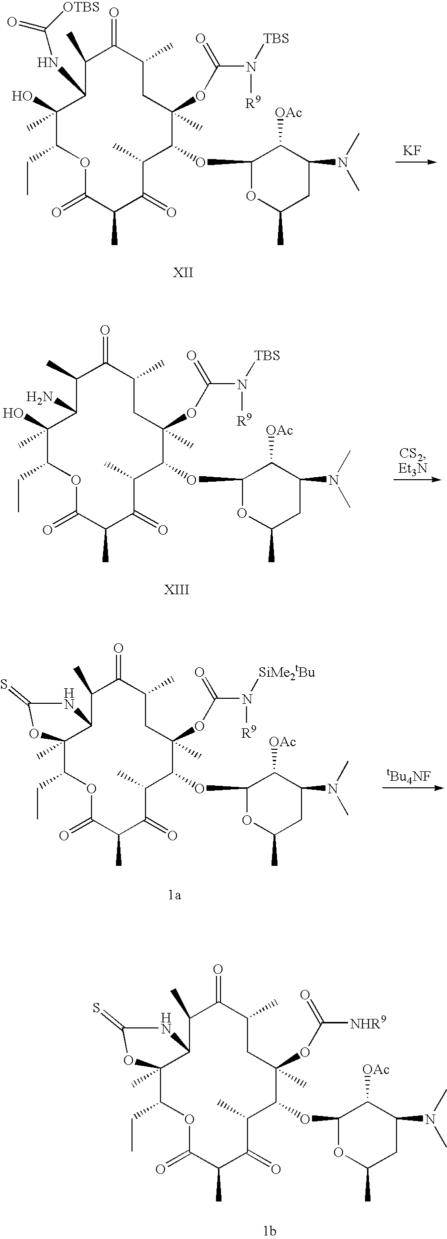

Compounds of formula IX, wherein R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, can be obtained by selective acylation of the cyclic carbamate of compounds of formula VIII with di-tert-butyl dicarbonate in the presence of a catalytic amount dimethylaminopyridine (DMAP), in an inert solvent such as THF or methylene chloride. Typically, the reaction is conducted for from 0.5-24 hours at 0° C. to 80° C. Compounds of formula VIII, wherein R⁹ is hydrogen, can be prepared as described in Scheme 1. Compounds of formula VIII, wherein R⁹ is optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, can be prepared by methods described in WO 02/46204. Hydrolysis of the 11,12-cyclic carbamate of IX can be conducted with a base, such as lithium hydroxide, sodium hydroxide, potassium hydroxide, sodium carbonate or potassium carbonate, in an aqueous solvent mixture such as methanol/water, THF/water or THF/methanol/water to give compounds of formula X. Typically, this reaction is conducted for from 2 hours to 10 days at temperatures ranging from 0° C. to 80° C. Under the reaction conditions, the 2′-hydroxyl protecting group may also undergo hydrolysis. Reprotection of the 2′-hydroxyl group of X can be carried out by treatment with acetic anhydride in the presence of a tertiary amine base, such as triethylamine, diisopropylethylamine, or pyridine, and optionally an acylation catalyst, such as DMAP, in a suitable solvent such as methylene chloride, chloroform or THF at a temperature ranging from −20° C. to 37° C. for 2 to 48 hours. It is understood that alternate protecting groups may be employed for the 2′-hydroxyl functionality of the desosamine sugar by treatment of X with reagents such as benzoic anhydride, benzyl chloroformate, hexamethyldisilazane, or a trialkylsilyl chloride. Oxidation of the 3-hydroxyl group to give compounds of formula XI can be carried out by treatment with Dess-Martin periodinane in an inert solvent such as methylene chloride or chloroform. Typically, the reaction is conducted for from 0.5-48 hours at 0° C. to room temperature. Alternative methods of oxidation include using dimethyl sulfoxide (DMSO) and a carbodiimide, such as DCC or EDCI, in the presence of a pyridinium salt, such as pyridinium trifluoroacetate in an inert solvent such as methylene chloride or THF, or N-chlororsuccinimide and dimethylsulfoxide complex followed by treatment with a tertiary amine base. The 11-tert-butyl carbamate functionality of XI can be transformed to the corresponding 11-tert-butyldimethylsilyl (TBS) carbamate XII by treatment with tert-butyldimethylsilyl trifluoromethanesulfonate, in the presence of a base such as 2,6-lutidine or pyridine, in an inert solvent, such as methylene chloride, chloroform or THF for 1-48 hours at a temperature ranging from −20° C. to room temperature. The C6-carbamate of compounds of formula XI may also be silylated under these conditions. Alternative silylating agents, such as trimethylsilyl trifluoromethanesulfonate, triethylsilyl trifluoromethanesulfonate or triisopropylsilyl trifluoromethanesulfonate may likewise be used to convert the 11-tert-butyl carbamate of XI to other 11-trialkylsilyl carbamates, such as the 11-trimethylsilyl carbamate, 11-triethylsilyl carbamate or 11-triisopropylsilyl carbamate, respectively. Deprotection of the 11-amino functionality of XII can be effected by reaction with a fluoride salt, such as sodium fluoride, potassium fluoride or cesium fluoride, in a solvent such as THF, MeOH or DMF. Typically, the reaction is carried out for from 0.5-24 hours at temperatures ranging from 0° C. to 80° C. Treatment of the resulting 11-amino, 12-hydroxy compounds of formula XIII with carbon disulfide (CS₂) gives 11,12-cyclic thiocarbamate compounds 1a. Typically, the reaction is conducted in the presence of a tertiary amine base, such as triethylamine, diisopropylethylamine, or pyridine, and optionally an acylation catalyst, such as DMAP, in a suitable solvent such as THF, methylene chloride, or DMF at a temperature ranging from 0° C. to 100° C. for 2 to 48 hours. Finally, the tert-butyldimethylsilyl group on the C6-carbamate of 1a can be removed by treatment with an ammonium fluoride salt, such as tetrabutylammonium fluoride, to give compounds of formula 1b. The reaction is typically carried out in a suitable solvent, such as THF or dioxane, at temperatures ranging from 0° C. to 100° C. for from 0.5-24 hours.

An alterative method to synthesize compounds of formula 1b from XII is outlined in Scheme 3.

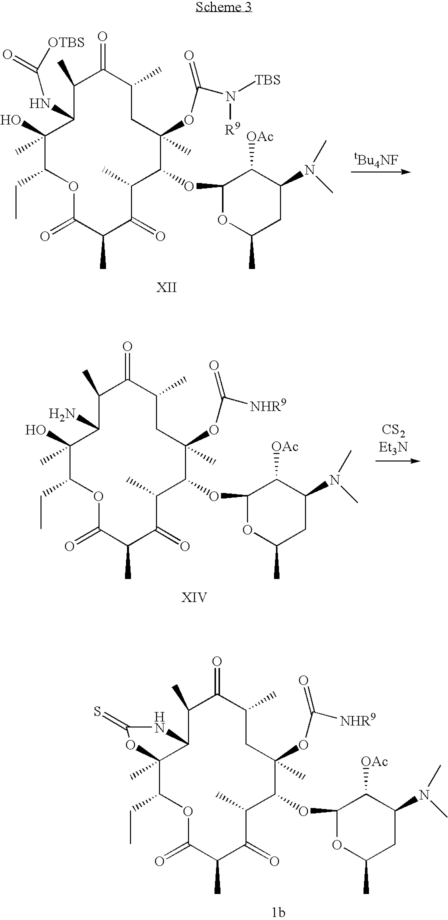

Compounds of formula XII are treated with an ammonium fluoride salt, such tetrabutylammonium fluoride, in a suitable solvent such as THF or dioxane, to remove both tert-butyldimethylsilyl groups and give compounds of formula XIV. Typically, the reaction is conducted at temperatures ranging from 0° C. to 100° C. for from 0.5-24 hours. Compounds of formula XIV can be converted to the 11,12-thiocarbamate compounds 1b using reaction conditions described above.

An alternate method to synthesize compounds of formula XI is outlined in Scheme 4.

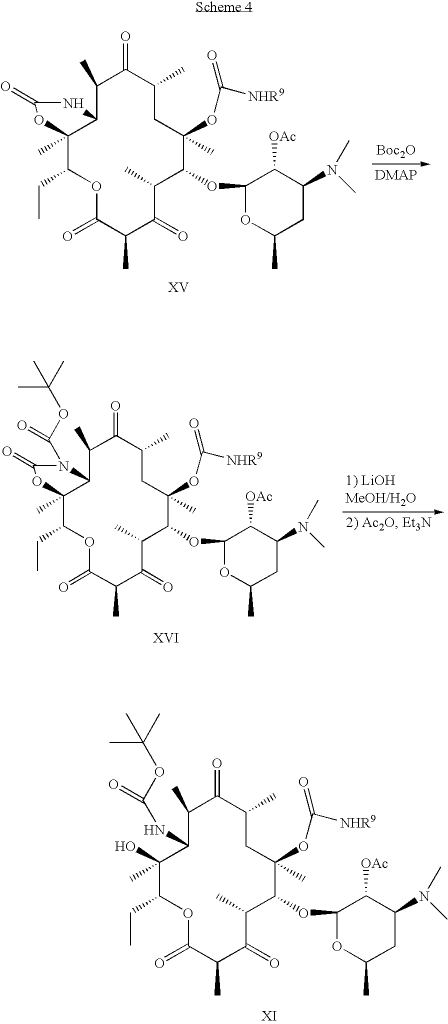

Compounds of formula XV, wherein R⁹ is hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, can be prepared by methods described in WO 02/46204. Selective acylation of the cyclic carbamate of compounds of formula XV is effected with di-tert-butyl dicarbonate in the presence of a catalytic amount of dimethylaminopyridine (DMAP), in an inert solvent such as THF or methylene chloride to give compounds of formula XVI. Typically, the reaction is conducted for from 0.5-24 hours at 0° C. to 80° C. Hydrolysis of the 11,12-cyclic carbamate of XVI can be conducted with a base, such as lithium hydroxide, sodium hydroxide, potassium hydroxide, sodium carbonate or potassium carbonate, in an aqueous solvent mixture such as methanol/water, THF/water or THF/methanol/water. Typically, this reaction is conducted for from 2 hours to 10 days at temperatures ranging from 0° C. to 80° C. Under the reaction conditions, the 2′-hydroxyl protecting group may also undergo hydrolysis. Reprotection of the 2′-hydroxyl group can be carried out by treatment with acetic anhydride in the presence of a tertiary amine base, such as triethylamine, diisopropylethylamine, or pyridine, and optionally an acylation catalyst, such as DMAP, in a suitable solvent such as methylene chloride, chloroform or THF at a temperature ranging from −20° C. to 37° C. for 2 to 48 hours to give compounds of formula XI.

Scheme 5 depicts the synthesis of compounds of formulae XVIII, XIX, XX, XXI, XXII, 1c, and 1d, wherein E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl using similar methods as described in Scheme 2. Compounds of formula XVII, prepared as described in WO 98/09978,) are treated with di-tert-butyl dicarbonate in the presence of catalytic amount dimethylaminopyridine (DMAP), in an inert solvent such as THF or methylene chloride. Typically, the reaction is conducted for from 0.5-24 hours at 0° C. to 80° C. Hydrolysis of the 11,12-cyclic carbamate of XVIII can be conducted using a base, such as lithium hydroxide, sodium hydroxide, potassium hydroxide, sodium carbonate or potassium carbonate, in an aqueous solvent mixture such as methanol/water, THF/water or THF/methanol/water. Typically, this reaction is conducted for from 2 hours to 10 days at temperatures ranging from 0° C. to 80° C. Under the reaction conditions, the 2′-hydroxyl protecting group may also undergo hydrolysis. Reprotection of the 2′-hydroxyl group by treatment with acetic anhydride in the presence of a tertiary amine base, such as triethylamine, diisopropylethylamine, or pyridine, and optionally an acylation catalyst, such as DMAP, leads to compound XIX. The reaction is typically carried out in a suitable solvent such as methylene chloride, chloroform or THF at a temperature ranging from −20° C. to 37° C. for 2 to 48 hours. It is understood that alternative protecting groups may be employed for the desosamine sugar by reaction of the 2′hydroxyl functionality with reagents such as benzoic anhydride, benzyl chloroformate, hexamethyldisilazane, or a trialkylsilyl chloride. The tert-butoxy carbonyl groups of XIX are removed using similar methods as described in Scheme 2. Treatment of compound XIX with tert-butyldimethylsilyl trifluoromethanesulfonate followed by potassium fluoride leads to compound XX. Compound XX may be converted into compounds of formula XXI by reaction with an acylating agent optionally in the presence of an amine base, such as pyridine, triethylamine or diisopropylethylamine, in an inert solvent such as dichloromethane, tetrahydrofuran or toluene at temperatures ranging from −20° C. to 60° C. for from 1-48 hours. Acylating agents include acid halides, acid anhydrides, and acids in the presence of an activating agent such as dicyclohexylcarbodiimide, EDCI, BOP—Cl, BOP, PyBOP, and the like. Compound XX may be converted also to compounds of formula XXII by reaction with an aldehyde RCHO (R may be a member of the group including, but not limited to, hydrogen, alkyl, alkenyl, alkynyl, cycloalkyl, aryl, heteroaryl, heterocycle, arylalkenyl, arylalkynyl, aralkyl, heteroarylalkenyl, heteroarylalkynyl, heteroarylalkyl, heterocycloalkenyl, heterocycloalkynyl, and heterocycloalkyl).in the presence of a suitable reducing agent, such as sodium cyanoborohydride, and an acid catalyst, such as acetic acid at a temperature ranging from 0° C. to 60° C. for from 1 to 24 hours. Compounds of formulae XXI and XXII may be converted to compounds of the invention of formulae 1c and 1d, respectively, by oxidation of the 3-hydroxyl group, using methods previously described in Scheme 2, followed by removal of the 2′-acetyl protecting group. A preferred method for oxidation of the 3-hydroxyl group is treatment with Dess-Martin periodinane in an inert solvent such as methylene chloride or chloroform. Typically, the reaction is conducted for from 0.5-48 hours at 0° C. to room temperature. A preferred method for removal of the 2′-acetyl protecting group is by transesterification with methanol for from 2 to 72 hours at room temperature.

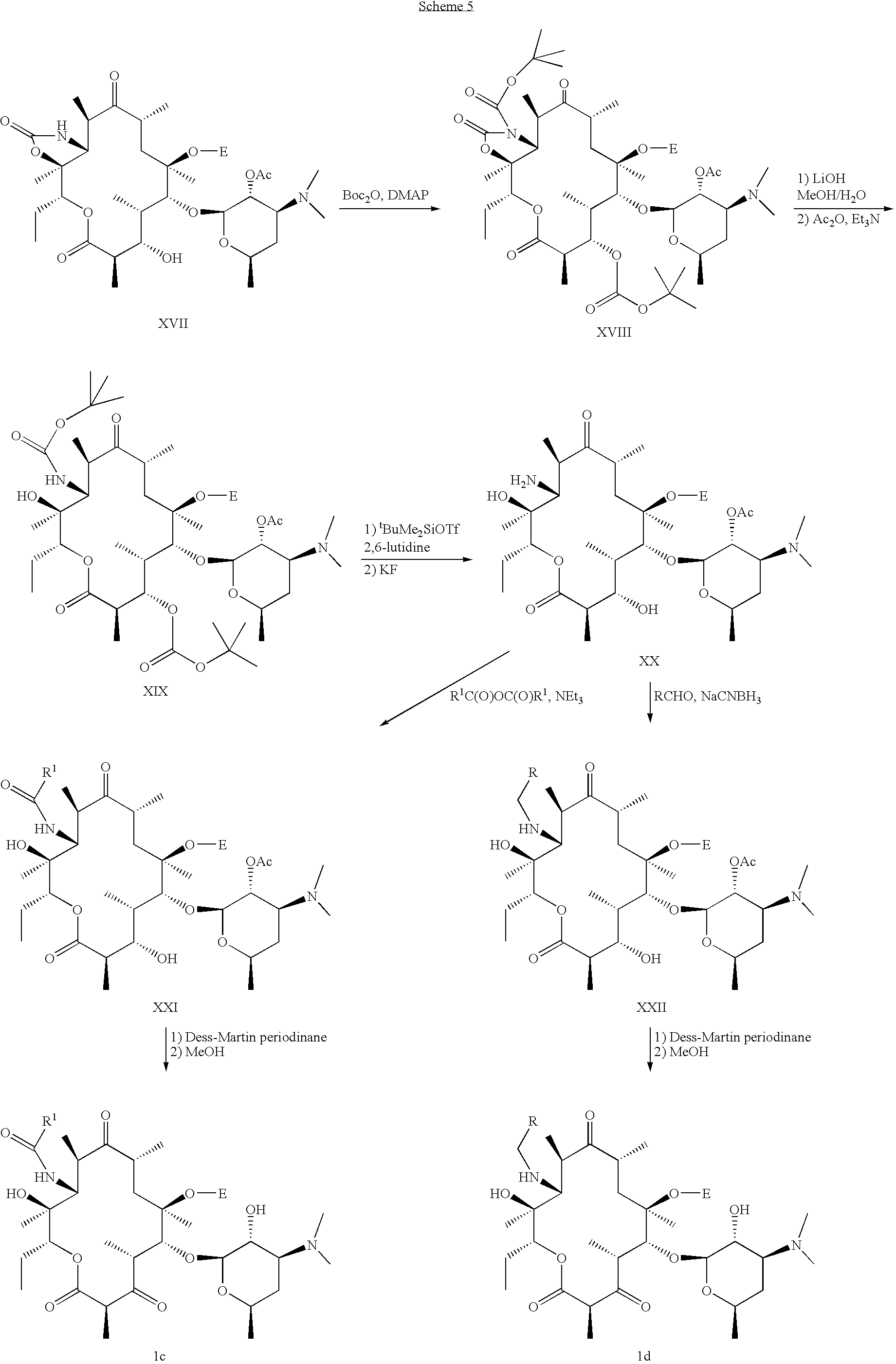

Scheme 6 depicts an alternate synthesis of compounds of formulae 1c and 1d beginning with compounds of formula XXIII, wherein E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl (prepared as described in WO 98/09978). Compounds of formula XXIII are treated with di-tert-butyl dicarbonate in the presence of catalytic amount dimethylaminopyridine (DMAP), in an inert solvent such as THF or methylene chloride. Typically, the reaction is conducted for from 0.5-24 hours at 0° C. to 80° C. Hydrolysis of the 11,12-cyclic carbamate of XXIV can be conducted using a base, such as lithium hydroxide, sodium hydroxide, potassium hydroxide, sodium carbonate or potassium carbonate, in an aqueous solvent mixture such as methanol/water, THF/water or THF/methanol/water. Typically, this reaction is conducted for from 2 hours to 10 days at temperatures ranging from 0° C. to 80° C. Under the reaction conditions, the 2′-hydroxyl protecting group may also undergo hydrolysis. Reprotection of the 2′-hydroxyl group by treatment with acetic anhydride in the presence of a tertiary amine base, such as triethylamine, diisopropylethylamine, or pyridine, and optionally an acylation catalyst, such as DMAP, leads to compound XXV. The reaction is typically carried out in a suitable solvent such as methylene chloride, chloroform or THF at a temperature ranging from −20° C. to 37° C. for 2 to 48 hours. It is understood that alternative protecting groups may be employed for the desosamine sugar by reaction of the 2′hydroxyl functionality with reagents such as benzoic anhydride, benzyl chloroformate, hexamethyldisilazane, or a trialkylsilyl chloride. The tert-butoxy carbonyl groups of XXV are removed using similar methods as described in Scheme 4. Treatment of compound XXV with tert-butyldimethylsilyl trifluoromethanesulfonate followed by potassium fluoride leads to compound XXVI. Compound XXVI may be converted into compounds of formula XXVII by reaction with an acylating agent optionally in the presence of an amine base, such as pyridine, triethylamine or diisopropylethylamine, in an inert solvent such as dichloromethane, tetrahydrofuran or toluene at temperatures ranging from −20° C. to 60° C. for from 1-48 hours. Acylating agents include acid halides, acid anhydrides, and acids in the presence of an activating agent such as dicyclohexylcarbodiimide, EDCI, BOP—Cl, BOP, PyBOP, and the like. Compound XXVI may be converted also to compounds of formula XXVIII by reaction with an aldehyde RCHO (R may be a member of the group including, but not limited to, hydrogen, alkyl, alkenyl, alkynyl, cycloalkyl, aryl, heteroaryl, heterocycle, arylalkenyl, arylalkynyl, aralkyl, heteroarylalkenyl, heteroarylalkynyl, heteroarylalkyl, heterocycloalkenyl, heterocycloalkynyl, and heterocycloalkyl).in the presence of a suitable reducing agent, such as sodium cyanoborohydride, and an acid catalyst, such as acetic acid at a temperature ranging from 0° C. to 60° C. for from 1 to 24 hours. Compounds of formulae XXVII and XXVIII may be converted to compounds of the invention of formulae 1c and 1d, respectively, by removal of the 2′-acetyl protecting group. A preferred method for removal of the 2′-acetyl protecting group is by transesterification with methanol for from 2 to 72 hours at room temperature.

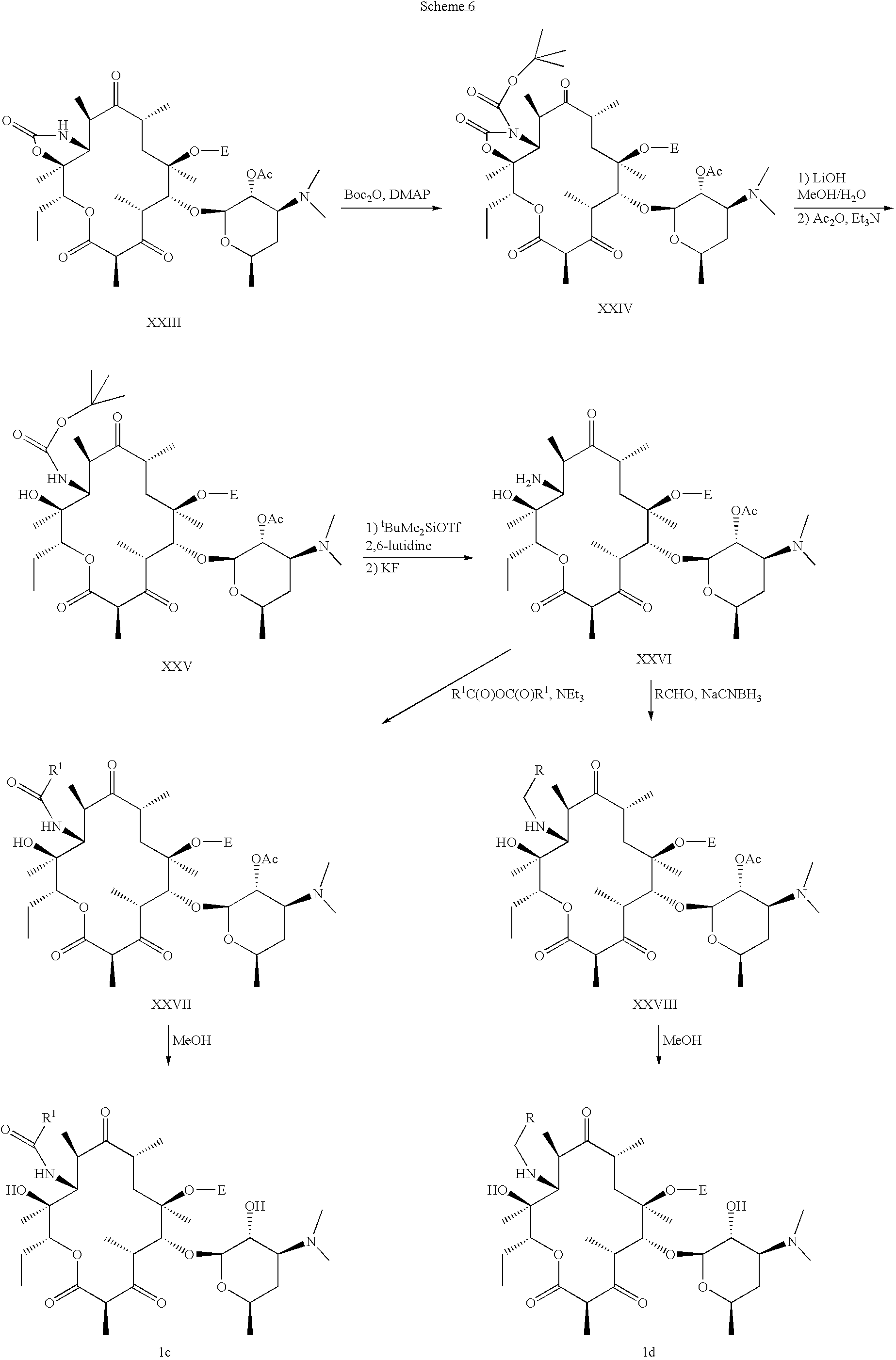

Scheme 7 depicts the synthesis of compounds of formulae XXIX, XXX, 1e, and 1f, wherein R⁹ is hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl. Compound XIV, prepared as described in Scheme 3, may be converted into compounds of formula XXIX by reaction with an acylating agent optionally in the presence of an amine base, such as pyridine, triethylamine or diisopropylethylamine, in an inert solvent such as dichloromethane, tetrahydrofuran or toluene at temperatures ranging from −20° C. to 60° C. for from 1-48 hours. Acylating agents include acid halides, acid anhydrides, and acids in the presence of an activating agent such as dicyclohexylcarbodiimide, EDCI, BOP—Cl, BOP, PyBOP, and the like. Compound XIV may be converted also to compounds of formula XXX by reaction with an aldehyde RCHO (R may be a member of the group including, but not limited to, hydrogen, alkyl, alkenyl, alkynyl, cycloalkyl, aryl, heteroaryl, heterocycle, arylalkenyl, arylalkynyl, aralkyl, heteroarylalkenyl, heteroarylalkynyl, heteroarylalkyl, heterocycloalkenyl, heterocycloalkynyl, and heterocycloalkyl).in the presence of a suitable reducing agent, such as sodium cyanoborohydride, and an acid catalyst, such as acetic acid at a temperature ranging from 0° C. to 60° C. for from 1 to 24 hours. Compounds of formulae XXIX and XXX may be converted to compounds of the invention of formulae 1e and 1f, respectively, by removal of the 2′-acetyl protecting group. A preferred method for removal of the 2′-acetyl protecting group is by transesterification with methanol for from 2 to 72 hours at room temperature.

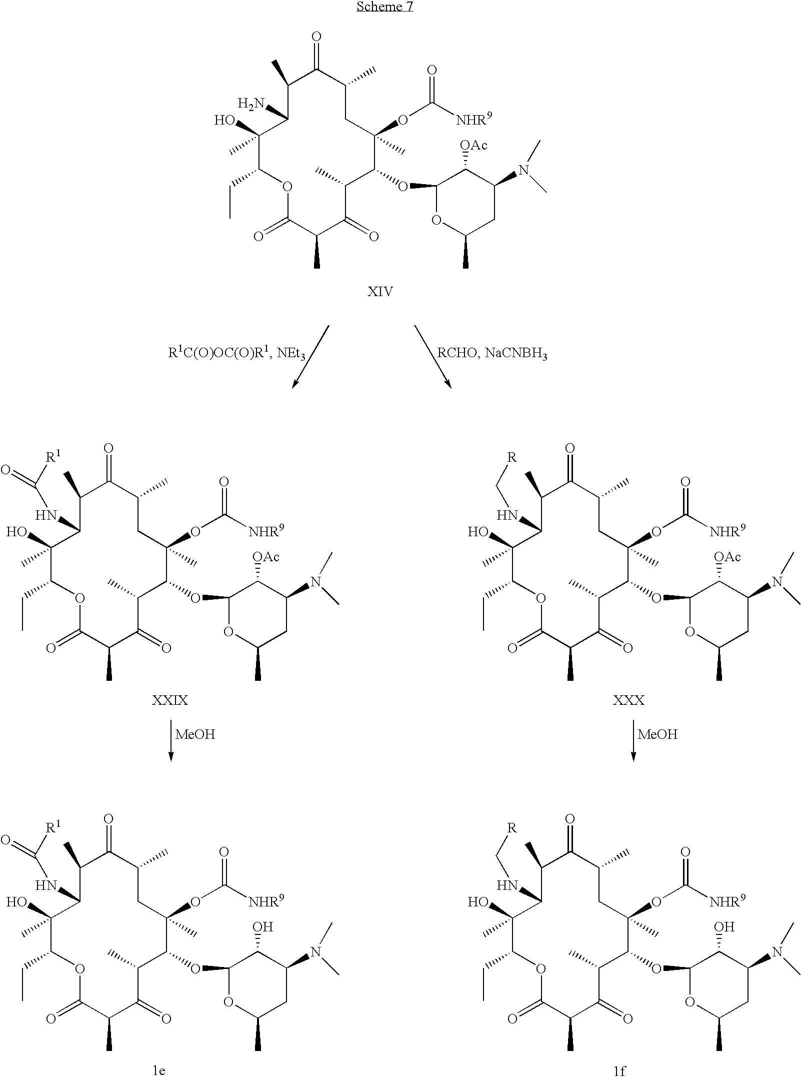

Scheme 8 illustrates the synthesis of compounds of formulae 1h, 1i, and 1j wherein RCHO is an aldehyde (R may be a member of the group including, but not limited to, hydrogen, alkyl, alkenyl, alkynyl, cycloalkyl, aryl, heteroaryl, heterocycle, arylalkenyl, arylalkynyl, aralkyl, heteroarylalkenyl, heteroarylalkynyl, heteroarylalkyl, heterocycloalkenyl, heterocycloalkynyl, and heterocycloalkyl). Selective acylation of the cyclic thiocarbamate of compound 1g (prepared as described in Scheme 2, 1b wherein R⁹ is hydrogen) with acetic anhydride leads to compound 1h. Typically, the reaction is conducted in the presence of a tertiary amine base, such as triethylamine, diisopropylethylamine, or pyridine, and optionally an acylation catalyst, such as DMAP, in a suitable solvent such as methylene chloride, chloroform or THF at a temperature ranging from 0° C. to 60° C. for 2 to 72 hours. Alternate acylating agents may also be used in the reaction (i.e., R¹C(O)OC(O)R¹ wherein R¹ is other than methyl) to provide compounds of formula 1h, wherein R¹ is as defined above. Compounds of the invention 1i can be obtained by alkylation of the primary carbamate of 1h with a suitably substituted aldehyde in the presence of a reducing agent and acid. Alternatively, the corresponding acetal may be used in place of the aldehyde. Preferred reagents for effecting this transformation are triethylsilane and trifluoroacetic acid in a suitable solvent, like acetonitrile, methylene chloride, or toluene at −20° C. to 100° C. Typically, the reaction is conducted for from 2-96 hours depending on the reactivity of the aldehyde or acetal. Removal of the 11-N and 2′-acetyl groups of compound 1i is readily accomplished by treatment with a base, such as lithium hydroxide, sodium hydroxide, potassium hydroxide, sodium carbonate or potassium carbonate, in a suitable solvent or solvent mixture such as methanol, methanol/water, THF/water or THF/methanol/water for from 16-72 hours preferably at room temperature to give compounds of the invention 1j.

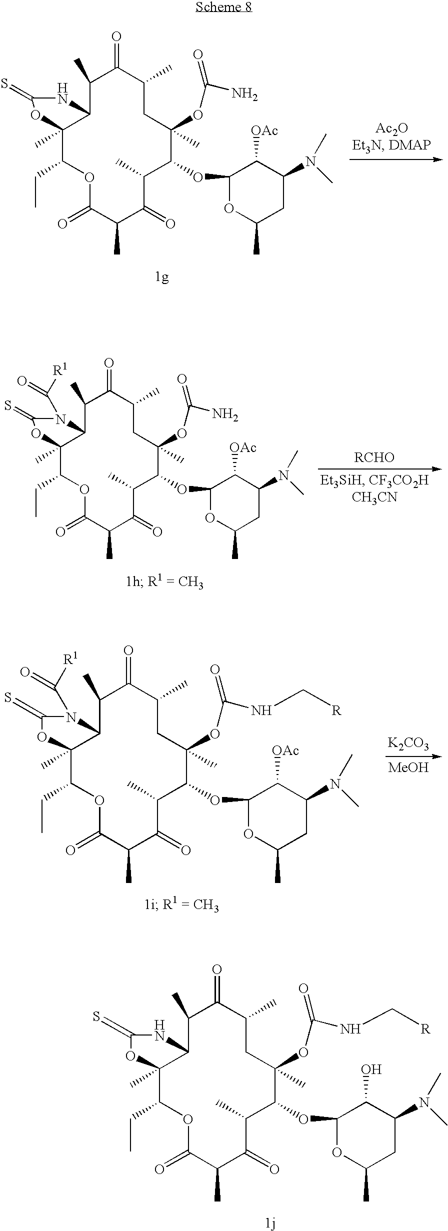

Scheme 9 depicts the synthesis of compounds of formulae 1k, 1l and 1m. Compounds of the invention 1k can be obtained by reaction of 1h with a suitably substituted 1,4-dialdehyde or its equivalent in the presence of an acid. Equivalents of 1,4-dialdehydes include 2,5-dialkyltetrahydrofurans, 1,4-dialdehyde monoacetals, and 1,4-dialdehyde diacetals. A preferred 1,4-dialdehyde equivalent is 2-formyl-4,4-dimethoxybutyronitrile. A preferred acid for effecting this transformation is trifluoroacetic acid in a suitable solvent, like acetonitrile, methylene chloride, or toluene at −20° C. to 100° C. Typically the reaction is conducted for from 2 to 96 hours. Compounds of formula 1l can be prepared by reaction of 1k (preferably wherein R⁵═CN and R⁶═H) with a suitably substituted alcohol in the presence of a suitable base, such as DBU, DBN, tert-butyltetramethylguanidine, sodium hydride, potassium hydride, or an alkyllithium in a suitable solvent, such as acetonitrile, dimethylformamide, dimethylsulfoxide, or THF, at a temperature ranging from −20° C. to 120° C. for 0.5 to 72 hours. Preformed alkali or alkaline earth metal alkoxides are also suitable reagents for the preparation of compounds of formula 1l. It is understood that the 11-N protecting group may also be removed during the course of the reaction. Removal of the 2′-acetyl group of 1l is readily accomplished by transesterification with methanol for from 2 to 72 hours at room temperature to give compounds of the formula 1m.

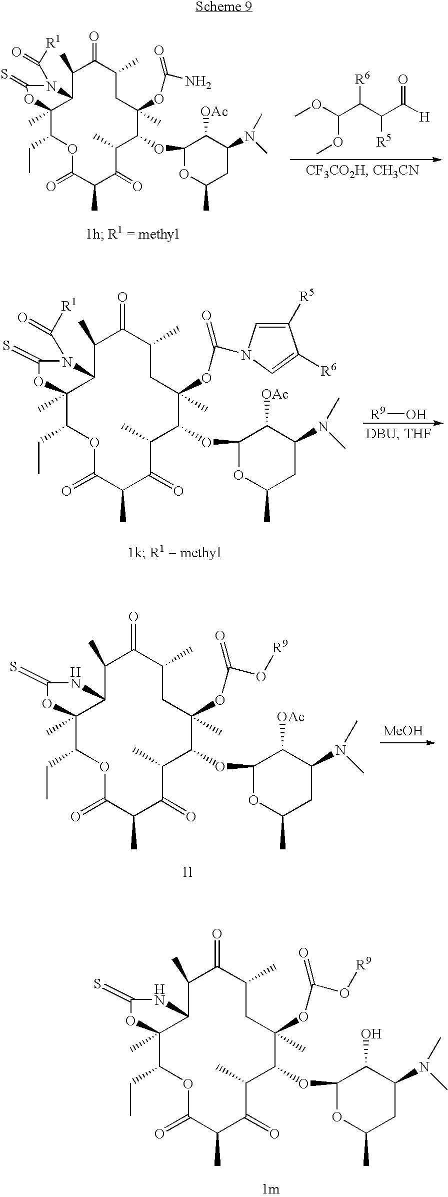

Scheme 10 illustrates a method for conversion of a compound of the invention containing an alkenyl functionality, such as substituted O-propenyl carbamate derivative 1n, to a compound of the invention containing an alkyl functionality, such as substituted O-propyl carbamate compound 1o. Typically, this transformation is conducted via catalytic transfer hydrogenation, in which the olefin is reacted with ammonium formate in the presence of a suitable catalyst, such as palladium on carbon, in a suitable solvent, such as methanol or ethanol, at a temperature ranging from 20° C. to 60° C. for 15 minutes to 24 hours. Other methods for reduction of the double bond could also be applicable, for example treatment with hydrogen in the presence of a noble metal catalyst, such as palladium or platinum. It will be obvious to one skilled in the art that the analogous O-propynylcarbamate may likewise be reduced to the corresponding O-propenylcarbamate or O-propylcarbamate under similar conditions.

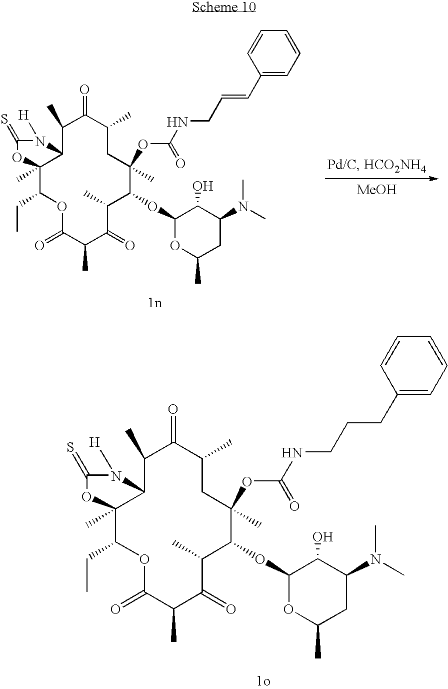

Scheme 11 illustrates the synthesis of certain aldehydes used in the preparation of compounds of the invention. In particular, a primary alcohol (XXXI) may be oxidized to the corresponding aldehyde (XXXII) using any of a number of methods known to those skilled in the art, including oxidation with pyridinium dichromate, pyridinium chlorochromate, tetrapropylammonium perruthenate and molecular oxygen, Dess-Martin periodinane, N-chlorosuccinimide-dimethylsulfide in the presence of a tertiary amine base, or oxalyl chloride-dimethylsulfoxide in the presence of a tertiary amine base. Typically, these reactions are conducted in an appropriate inert solvent, such as methylene chloride, chloroform, dichloroethane, benzene, toluene, or the like. A preferred oxidizing agent is Dess-Martin periodinane(1,1,1-tris(acetyloxy)-1,1-dihydro-1,2-benziodoxol-3(1H)-one) in methylene chloride for from 10 minutes to 48 hours at a temperature ranging from 0° C. to 37° C.

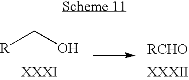

Scheme 12 also illustrates a method of synthesis of certain of the aldehydes (XXXIV) used in the preparation of compounds of the invention. Wittig-type reaction of an aromatic or heteroaromatic aldehyde (XXXIII) with 1,3-dioxolan-2-yl-methyltriphenylphosphonium bromide under phase transfer conditions in a biphasic solvent system in the presence of an inorganic base, such as potassium carbonate, affords the corresponding vinylogous aldehyde (XXXIV). The reaction is typically run from 2 to 48 hours at temperatures ranging from 0° C. to 37° C. The method is more fully described in Daubresse, N., Francesch, C. and Rolando, C., Tetrahedron, 1998, 54, 10761.

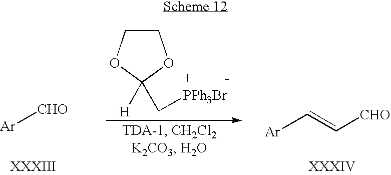

Scheme 13 also illustrates the synthesis of certain of the aldehydes (XXXVI) used in the preparation of compounds of the invention. Reaction of a bromocinnamaldehyde derivative (XXXV) with an aryl boronic acid to give the biaryl derivative (XXXVI) is conducted under typical Suzuki coupling conditions, i.e., in the presence of a Pd⁰ catalyst, typically palladium tetrakistriphenylphosphine, and a base, typically sodium carbonate, potassium carbonate, sodium bicarbonate, potassium bicarbonate, potassium phosphate, or triethylamine in a suitable solvent, such as toluene, ethanol, methanol, DME, or THF. Reaction time is typically 2 to 48 hours at a temperature ranging from 20° C. to 110° C. Aryl iodides and aryl triflates are also suitable substrates for this conversion.

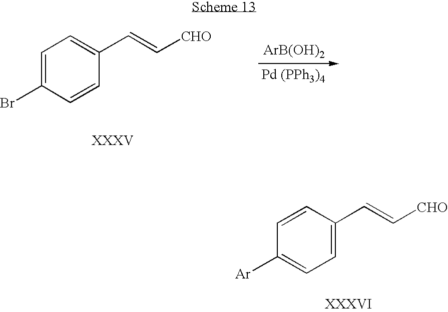

Scheme 14 illustrates a method for the preparation of certain alcohols (XXXVIII) used in the preparation of some of the compounds of the invention. In this method, an aldehyde XXXVII is reduced to the alcohol XXXVIII. A preferred reducing agent is sodium borohydride in an alcoholic solvent such as methanol or ethanol. Another preferred reducing agent is diisobutylaluminum hydride in an inert solvent such as dichloromethane, toluene, or tetrahydrofuran. It will be obvious to one skilled in the art that numerous methods for reducing an aldehyde to an alcohol are known, and any of these may be suitable provided the method is compatible with other functional groups that may be present in the molecule.

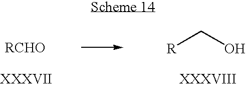

Scheme 15 also depicts a method of synthesis of certain alcohols (XL) used in the preparation of compounds of the invention. In this method, an ester XXXIX is reduced to the alcohol XL. Several methods to effect this transformation are known to one skilled in the art, including reduction with lithium aluminum hydride, lithium borohydride, diisobutylaluminum hydride, or sodium borohydride, among others. A preferred reducing agent is diisobutylaluminum hydride in an inert solvent such as dichloromethane, toluene, or tetrahydrofuran. Another preferred reducing agent is sodium borohydride in methanol, ethanol, or alternatively in tetrahydrofuran/methanol or tetrahydrofuran/ethanol mixtures.

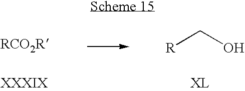

Scheme 16 illustrates the synthesis of certain alcohols (XLII) and aldehydes (XLIII) of the invention. Reaction of a suitably substituted aromatic or heteroaromatic aldehyde (XXXIII) with an activated phosphonate derivative, such as triethyl 2-fluoro2-phophonoacetate, optionally in the presence of magnesium bromide and a suitable base, provides the corresponding □,□-unstaurated ester (XLI). Alternate bases may be used to effect the transformation, including organolithium reagents, lithium diisopropylamide, potassium tert-butoxide, diazabicycloundecane, and the like. Typically, the reaction is conducted in an inert solvent, such as tetrahydrofuran, hexane, or a tetrahydrofuran/hexane mixture, at temperatures ranging from −78° C. to 60° C. for from 1 to 48 hours. Other methods may also be used to effect this conversion, such as reaction of an appropriately substituted aldehyde with the sodium salt of diethyl 2-oxo-3-fluorobutan-1,4-dioate in tetrahydrofuran. Reduction of the ester (XLI) to the corresponding alcohol (XLII) can be conducted as described in Scheme 15. A preferred method for executing this transformation is reduction with diisobutylaluminum hydride in an inert solvent such as dichloromethane, toluene, or tetrahydrofuran at a temperature ranging from −78° C. to room temperature for from 10 minutes to 24 hours. An alternate preferred method is reduction with sodium borohydride in a tetrahydrofuran/ethanol mixture at a temperature ranging from −20° C. to 25° C. for from 1 to 48 hours. Oxidation of alcohol XLII to the corresponding aldehyde (XLIII) is conducted as described in Scheme 11. A preferred method for executing this transformation is oxidation with Dess-Martin periodinane(1,1,1-tris(acetyloxy)-1,1-dihydro-1,2-benziodoxol-3(1H)-one) in methylene chloride for from 10 minutes to 48 hours at a temperature ranging from 0° C. to 37° C.

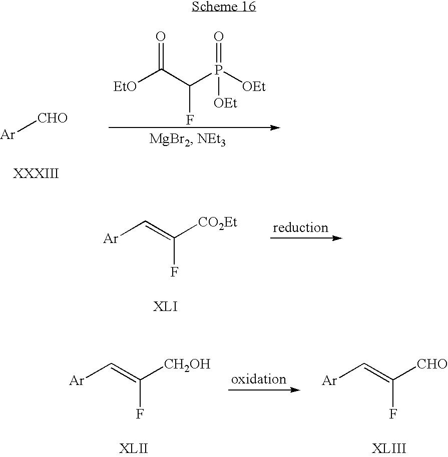

Scheme 17 depicts the synthesis of certain aldehydes used in the preparation of compounds of the invention. Reaction of an appropriately substituted aromatic or heteroaromatic Grignard reagent (XLIV) with an acrolein derivative, such as 3-dimethylaminoacrolein, provides the corresponding □,□-unsaturated aldehyde (XXXIV). Other substituted acrolein derivatives may also serve as the electrophile, including 3-methoxyacrolein, 3-ethoxyacrolein, 3-phenoxyacrolein, or 3-trimethylsilyloxyacrolein. Typically, the reaction is conducted in an inert solvent such as tetrahydrofuran, diethyl ether, or glyme at temperatures ranging from −78° C. to 25° C. for from 30 minutes to 24 hours.

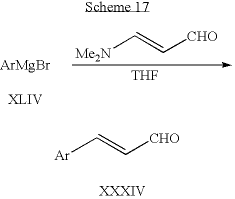

Scheme 18 illustrates the synthesis of certain of the propargyl alcohols (XLVII) used in the preparation of compounds of the invention. Reaction of halophenylboronic acid derivative (XLV) with propargyl alcohol to give the hydroxypropynylphenylboronic acid derivative (XLVI) is conducted in the presence of a Pd⁰ catalyst, typically palladium tetrakistriphenylphosphine, and pyrrolidine as solvent. Reaction time is typically from 2 to 48 hours at a temperature ranging from 0° C. to 85° C. Conversion of XLVI to the biarylpropargyl alcohol derivative (XLVII) is then conducted under Suzuki coupling conditions, i.e., by reaction with an aryl or heteroaryl bromide in the presence of a Pd⁰ catalyst, typically palladium tetrakistriphenylphosphine, and a base, typically sodium carbonate, potassium carbonate, sodium bicarbonate, potassium bicarbonate, potassium phosphate, or triethylamine in a suitable solvent, such as toluene, ethanol, methanol, DME, THF, water, or aqueous solvent mixtures. Reaction time is typically 2 to 48 hours at a temperature ranging from 20° C. to 110° C. Aryl iodides and aryl triflates are also suitable substrates for this conversion.

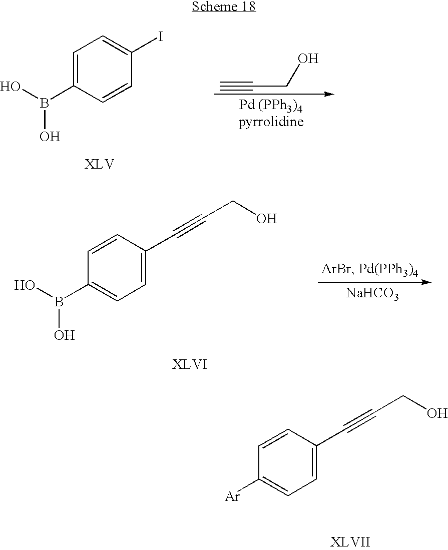

Compounds of the invention wherein R³ is a hydroxy protecting group other than acyl may be prepared by methods analogous to those shown in the above schemes with appropriate reagents that are either commercially available or may be made by known methods.

Compounds of the invention wherein R⁴ is a group other than ethyl may be prepared beginning with modified erythromycin derivatives as starting materials as described in various publications including, but not limited to, WO99/35157, WO00/62783, WO00/63224, and WO00/63225, which are all incorporated by reference herein.

Compounds of the invention, wherein R² is halogen or hydroxy, may be prepared by methods analogous to those described in WO 02/46204.

These compounds have antimicrobial activity against susceptible and drug resistant Gram positive and Gram negative bacteria. In particular, they are useful as broad spectrum antibacterial agents for the treatment of bacterial infections in humans and animals. These compounds are particularly active against S. aureus, S. epidermidis, S. pneumoniae, S. pyogenes, Enterococci, Moraxella catarrhalis and H. influenzae. These compounds are particularly useful in the treatment of community-acquired pneumonia, upper and lower respiratory tract infections, skin and soft tissue infections, meningitis, hospital-acquired lung infections, and bone and joint infections.

Minimum inhibitory concentration (MIC) has been an indicator of in vitro antibacterial activity widely used in the art. The in vitro antimicrobial activity of the compounds was determined by the microdilution broth method following the test method from the National Committee for Clinical Laboratory Standards (NCCLS). This method is described in the NCCLS Document M7-A4, Vol. 17, No.2, “Methods for Dilution Antimicrobial Susceptibility Test for Bacteria that Grow Aerobically—Fourth Edition”, which is incorporated herein by reference.

In this method two-fold serial dilutions of drug in cation adjusted Mueller-Hinton broth are added to wells in microdilution trays. The test organisms are prepared by adjusting the turbidity of actively growing broth cultures so that the final concentration of test organism after it is added to the wells is approximately 5×10⁴ CFU/well.

Following inoculation of the microdilution trays, the trays are incubated at 35 □C for 16-20 hours and then read. The MIC is the lowest concentration of test compound that completely inhibits growth of the test organism. The amount of growth in the wells containing the test compound is compared with the amount of growth in the growth-control wells (no test compound) used in each tray. As set forth in Table 1, compounds of the present invention were tested against a variety of Gram positive and Gram negative pathogenic bacteria resulting in a range of activities depending on the organism tested.

Tables 1 below set forth the biological activity (MIC, μg/mL) of some compounds of the present invention.

TABLE 1
MIC Values (μg/mL) of Some Compounds of Formula 1
(A: S. aureus ATCC29213; B: E. faecalis ATCC29212;
C: S. pneumoniae ATCC49619; D: H. influenzae ATCC49247)

| Compound No. | A | B | C | D |
|---|---|---|---|---|
| 3 | 1 | 0.25 | 0.12 | 8 |
| 4 | 0.5 | 0.25 | 0.12 | 2 |
| 5 | 0.25 | 0.06 | ≦0.015 | 1 |
| 6 | 0.5 | 0.06 | ≦0.015 | 1 |
| 7 | 0.5 | 0.06 | ≦0.015 | 1 |
| 9 | 0.12 | 0.06 | ND | 2 |
| 10 | 0.25 | 0.12 | ≦0.015 | 2 |
| 11 | 0.25 | 0.12 | 0.03 | 2 |
| 12 | 0.5 | 0.12 | 0.03 | 1 |
| 13 | 0.5 | 0.12 | 0.03 | 4 |

This invention further provides a method of treating bacterial infections, or enhancing or potentiating the activity of other antibacterial agents, in warm-blooded animals, which comprises administering to the animals a compound of the invention alone or in admixture with another antibacterial agent in the form of a medicament according to the invention.

When the compounds are employed for the above utility, they may be combined with one or more pharmaceutically acceptable carriers, e.g., solvents, diluents, and the like, and may be administered orally in such forms as tablets, capsules, dispersible powders, granules, or suspensions containing for example, from about 0.5% to 5% of suspending agent, syrups containing, for example, from about 10% to 50% of sugar, and elixirs containing, for example, from about 20% to 50% ethanol, and the like, or parenterally in the form of sterile injectable solutions or suspensions containing from about 0.5% to 5% suspending agent in an isotonic medium. These pharmaceutical preparations may contain, for example, from about 0.5% up to about 90% of the active ingredient in combination with the carrier, more usually between 5% and 60% by weight.

Compositions for topical application may take the form of liquids, creams or gels, containing a therapeutically effective concentration of a compound of the invention admixed with a dermatologically acceptable carrier.

In preparing the compositions in oral dosage form, any of the usual pharmaceutical media may be employed. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compounds is preferred. These active compounds may also be administered parenterally or intraperitoneally. Solutions or suspensions of these active compounds as a free base or pharmacological acceptable salt can be prepared in water suitably mixed with a surfactant such as hydroxypropyl-cellulose. Dispersions can also be prepared in glycerol, liquid polyethylene glycols and mixtures thereof in oils. Under ordinary conditions of storage and use, these preparations may contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The effective dosage of active ingredient employed may vary depending on the particular compound employed, the mode of administration and the severity of the condition being treated. However, in general, satisfactory results are obtained when the compounds of the invention are administered at a daily dosage of from about 0.1 mg/kg to about 400 mg/kg of animal body weight, which may be given in divided doses two to four times a day, or in sustained release form. For most large mammals the total daily dosage is from about 0.07 g to 7.0 g, preferably from about 100 mg to 2000 mg. Dosage forms suitable for internal use comprise from about 100 mg to 1200 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The production of the above-mentioned pharmaceutical compositions and medicaments is carried out by any method known in the art, for example, by mixing the active ingredients(s) with the diluent(s) to form a pharmaceutical composition (e.g. a granulate) and then forming the composition into the medicament (e.g. tablets).

The following examples describe in detail the chemical synthesis of representative compounds of the present invention. The procedures are illustrations, and the invention should not be construed as being limited by chemical reactions and conditions they express. No attempt has been made to optimize the yields obtained in these reactions, and it would be obvious to one skilled in the art that variations in reaction times, temperatures, solvents, and/or reagents could increase the yields.

### EXAMPLE 1

### Compound VII

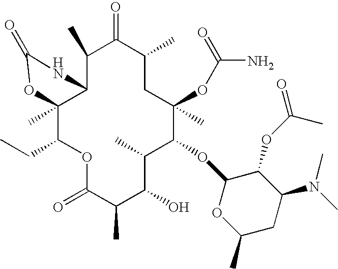 Step A

Triethylamine (42.0 mL, 301 mmol), DMAP (0.6 g, 4.9 mmol), and acetic anhydride (28.5 mL, 302 mmol) were added to a 0° C. suspension of erythromycin (36.7 g, 50 mmol) in dichloromethane (250 mL). The mixture was allowed to warm to room temperature and stir for 18 h. Methanol (10 mL) was added and stirring was continued for 5 min. The mixture was diluted with ether (750 mL), washed with sat. aq. NaHCO₃, water, and brine (500 mL each), dried (MgSO₄), and concentrated to provide compound I compound as a colorless foam. The material was used in the next step without further purification. MS 860 (M+H)⁺.

Step B

Sodium hexamethyldisilazide (1.0M in THF, 60.0 mL, 60.00 mmol) was added over 25 min to a 0° C. solution of I from step A (50.0 mmol) in THF (500 mL). After 2 h at 0° C., the mixture was diluted with water (250 mL) and brine (250 mL) and extracted with ethyl acetate (3×250 mL). The combined organic layers were dried (MgSO₄) and concentrated. The crude compound II was used in the next step without further purification. If desired, pure material could be obtained by chromatography (SiO₂, 95:5:0.2 dichloromethane/methanol/conc. NH₄OH). MS 800 (M+H)⁺.

Step C

Trichloroacetylisocyanate (13.4 mL, 112.5 mmol) was added over 20 min to a 0° C. solution of II from step B (37.5 mmol) in dichloromethane (250 mL). After 3 h at 0° C., the reaction was quenched by addition of methanol (20 mL) and concentrated to give crude compound III. The residue was dissolved in a mixture of methanol (250 mL), water (30 mL), and triethylamine (13 mL), heated to reflux for 2 h, and concentrated. The residue was dissolved in ethyl acetate (300 mL), washed with sat. aq. NaHCO₃ (200 mL) and brine (200 mL), dried (MgSO₄), and concentrated. The resulting mixture of C-10 epimers (IV) was dissolved in THF (250 mL) at 0° C. and potassium t-butoxide (1.0 M in THF, 47.0 mL, 47.0 mmol) was added over 15 min. The resulting mixture was stirred at 0° C. to 15° C. for 6 h. Sat. aq. NaHCO₃ (200 mL) was added, the bulk of the THF was removed in vacuo, and the resulting solution was extracted with ethyl acetate (3×250 mL). The combined organic extracts were washed with brine (200 mL), dried (MgSO₄), and concentrated. The crude compound V was used in the next step without further purification. MS 844 (M+H)⁺.

Step D

A solution of V from step C (37.5 mmol), triethylamine (11.2 mL, 80.57 mmol), and acetic anhydride (7.6 mL, 80.57 mmol) in dichloromethane (200 mL) was stirred at room temperature for 6 h. The solution was washed with sat. aq. NaHCO₃ (200 mL) and brine (200 mL), dried (MgSO₄), and concentrated. The crude compound VI was used in the next step without further purification. MS 886 (M+H)⁺.

Step E

Compound VI from step D (37.5 mmol) was dissolved in 1.2 N HCl (330 mL) and ethanol (100 mL) and stirred at room temperature for 24 h. The mixture was cooled to 0° C., made basic (pH>10) with 50% NaOH, and extracted with ethyl acetate (3×200 mL). The combined organic layers were washed with brine (200 mL), dried (MgSO₄), and concentrated. Purification by chromatography (SiO₂, 95:5:0.3 dichloromethane/methanol/conc. NH₄OH) yielded 14.1 g (55% based on erythromycin) of the title compound (VII) as a colorless solid. MS 686 (M+H)⁺.

### EXAMPLE 2

### Compound 1 (Formula 1b: R⁹ is hydrogen)

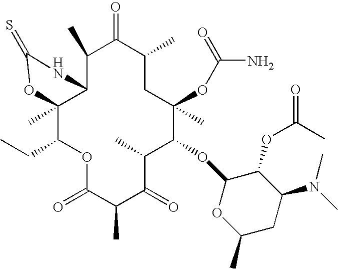 Step A

Compound VII from Example 1, step E (5.0 g, 7.3 mmole) was treated with di-tert-butyl dicarbonate (3.2 g, 14.6 mmole) and DMAP (0.27 g, 2.2 mmole) in THF (50 ml) at room temperature for 2 hours. The mixture was quenched with H₂O and extracted with EtOAc (3×). The combined organic layers were washed with 10% NH₄Cl, sat. aq. NaHCO₃, brine, dried (MgSO₄), and concentrated. Purification by chromatography (SiO₂, 95:5:0.3 dichloromethane/methanol/conc. NH₄OH) yielded 3.7 g (64 %) of compound VIII as a colorless solid. MS 786 (M+H)⁺

Step B

Compound VIII from step A (3.7 g, 4.7 mmole) was treated with LiOH (3.0 g, 125 mmole) in methanol/THF/H₂O (1:1:1, 30 mls) for 6 hours. The mixture was diluted with H₂O and extracted with EtOAc (3×). The combined organic layers were washed with H₂O, brine, dried (MgSO₄), and concentrated. Purification by chromatography (SiO₂, 95:5:0.3 dichloromethane/methanol/conc. NH₄OH) yielded 2.75 g (82%) of compound IXt as a colorless solid. MS 718 (M+H)⁺

Step C

Compound IX from step B (2.75 g, 3.8 mmole) was treated with acetic anhydride (0.72 mls, 7.7 mmole) and triethylamine (1.1 mls, 7.7 mmole) in CH₂Cl₂ for 2 hours. The mixture was washed with NaHCO₃, brine, dried and concentrated to give 3.0 g (quantitative) of the 2′-acetylated product as a colorless solid. MS 760 (M+H)⁺

Step D

The compound from step C (3.0 g, 3.948 mmole) was treated with Dess/Martin reagent (3.8 g, 8.9 mmole) and pyridine (1.6 mls, 19.7 mmole) in CH₂Cl₂ for 45 minutes. The mixture was quenched with NaHCO₃ and extracted with CH₂Cl₂ (3×). The combined organic layers were washed with NH₄Cl, NaHCO₃, brine, dried, and concentrated. Purification by chromatography (SiO₂, 95:5:0.3 dichloromethane/methanol/conc. NH₄OH) yielded 1.3 g (44.4%) of compound X as a colorless solid. MS 758 (M+H)⁺

Step E

Compound X from step D (1.3 g, 1.715 mmole) was treated with 2,6-lutidine (1.0 ml, 8.6 mmole) and tert-butyldimethylsilyltriflate (1.0 ml, 4.3 mmole) in CH₂Cl₂ at 0° C. for 45 minutes. The mixture was quenched with NaHCO₃ then extracted with CH₂Cl₂ (3×). The combined organic layers were washed with brine, dried (MgSO4), and concentrated to give 1.3 g (79%) of compound XI as a light yellow solid. MS 931 (M+H)⁺

Step F

Compound XI from step E (1.4 g, 1.5 mmole) was treated with KF (0.17 g, 3.0 mmole) in THF (20 mls) for 10 minutes. The mixture was quenched with H₂O and extracted with EtOAc (3×). The combined organic layers were washed with brine, dried (MgSO₄), and concentrated. Purification by chromatography (SiO₂, 95:5:0.3 dichloromethane/methanol/conc. NH₄OH) yielded 0.58 g (50%) of compound XII as a colorless solid. MS 772 (M+H)⁺

Step G

Compound XII from step F (0.47 g, 0.61 mole) was treated with carbon disulfide (0.3 mls, 4.87 mmole), triethylamine (0.85 mls, 6.1 mmole) in THF (20 mls) at 65° C. for 22 hours. The mixture was cooled, diluted with EtOAc, washed with NH₄Cl, washed with brine, dried, and concentrated to give 0.46 g (92%) of compound 1a as a light yellow solid. MS 814 (M+H)⁺

Step H

Compound 1a from step G (0.46 g, 0.56 mmole) was treated with 1.0 M (THF) tetrabutylammonium fluoride (1.1 mls, 1.1 mmole) in THF (20 mls) at 0° C. for 30 minutes. The mixture was concentrated in vacuo at 0° C. Purification by chromatography (SiO₂, 95:5:0.3 dichloromethane/methanol/conc. NH₄OH) yielded 0.11 g (28 %) of the title compound as a colorless solid. MS 700 (M+H)⁺

### EXAMPLE 3

### Compound 2 (Formula 1h)

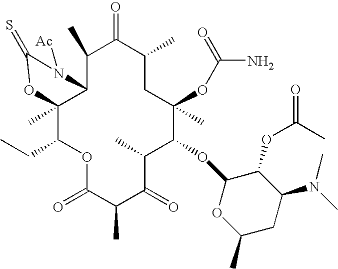

Compound 1 (0.1 g, 0.14 mmole) was treated with acetic anhydride (27 μL, 0.29 mmole), triethylamine (40 μL, 0.29 mmole) and a catalytic amount of DMAP in CH₂Cl₂ for 2 hours. The mixture was washed with NaHCO₃, brine, dried (MgSO₄), and concentrated to yield 0.11 g (quantitative) of the title compound as a colorless solid. MS 742 (M+H)⁺

### EXAMPLE 4

### Compound 3 (Formula 1j: R is (E)-[4-(2-pyrazinyl)phenyl]vinyl)

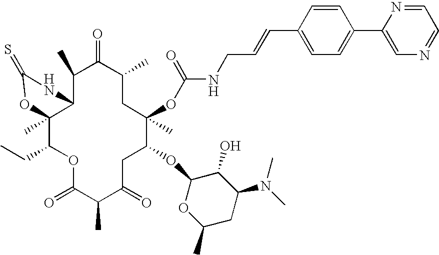 Step A

Compound 2 (40 mg, 0.054 mmole) and (2E)-3-[4-(2-pyrazinyl)phenyl]-2-propenal (34 mg, 0.16 mmole) were treated with triethylsilane (60 μL, 0.38 mmole) and trifluoroacetic acid (29 μL, 0.38 mmole) in acetonitrile (2.0 mls) at 65° C. in a sealed vessel for 17 hours. The mixture was cooled, quenched with NaHCO₃ and extracted with EtOAc. The combined organic layers were washed with brine, dried (MgSO₄), and concentrated. Purification by chromatography (SiO₂, 95:5:0.3 dichloromethane/methanol/conc. NH₄OH) yielded 33 mg (59%) of the compound of formula 1i wherein R is (E)-[4-(2-pyrazinyl)phenyl]vinyl. MS 936 (M+H)⁺

Step B

The compound from step A (33 mg, 0.035 mmole) was treated with K₂CO₃ (30 mg, 0.22 mmole) in methanol (2 ml) for 1 hour. The mixture was diluted with EtOAc, washed with H₂O, brine, dried (MgSO₄), concentrated, dissolved in methanol (2 ml) and stirred for 18 hours. The mixture was concentrated and purified by chromatography (SiO₂, 97:3:0.3 dichloromethane/methanol/conc. NH₄OH) to yield 10 mg (33%) of the title compound as a colorless solid. MS 852 (M+H)⁺

### EXAMPLE 5

### Compound 4 (Formula 1j: R is (E)-[4-(3-pyridazinyl)phenyl]vinyl)

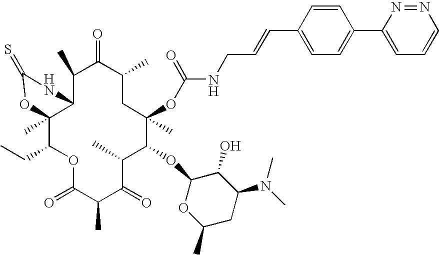 Step A

Compound 2 (53 mg, 0.071 mmole) and (2E)-3-[4-(3-pyridazinyl)phenyl]-2-propenal (45 mg, 0.21 mmole) were treated with triethylsilane (80 μL, 0.50 mmole) and trifluoroacetic acid (39 μL, 0.50 mmole) in acetonitrile (2.0 mls) at 65° C. in a sealed vessel for 18 hours. The mixture was cooled, quenched with NaHCO₃ and extracted with EtOAc. The combined organic layers were washed with brine, dried (MgSO₄), and concentrated. Purification by chromatography (SiO₂, 95:5:0.3 dichloromethane/methanol/conc. NH₄OH) yielded 35 mg (52%) of the compound of formula 1i wherein R is (E)-[4-(3-pyridazinyl)phenyl]vinyl. MS 936 (M+H)⁺

Step B

The compound from step A (35 mg, 0.037 mmole) was treated with K₂CO₃ (30 mg, 0.22 mmole) in methanol (2 ml) for 1 hour. The mixture was diluted with EtOAc, washed with H₂O, brine, dried (MgSO₄), concentrated, dissolved in methanol (2 ml) and stirred for 18 hours. The mixture was concentrated and purified by chromatography (SiO₂, 97:3:0.3 dichloromethane/methanol/conc. NH₄OH) to yield 19 mg (59%) of the title compound as a colorless solid. MS 852 (M+H)⁺

### EXAMPLE 6

### Compound 5 (Formula 1j: R is (E)-(3-quinolinyl)vinyl)

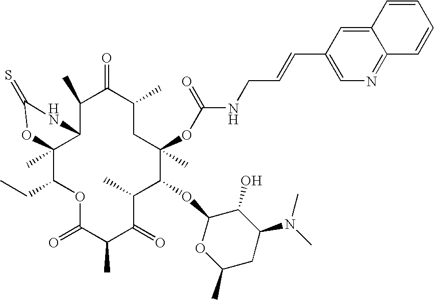 Step A

Compound 2 (50 mg, 0.067 mmole) and (2E)-3-(3-quinolinyl)-2-propenal (37 mg, 0.20 mmole) were treated with triethylsilane (75 μL, 0.47 mmole) and trifluoroacetic acid (36 μL, 0.47 mmole) in acetonitrile (2.0 mls) at 65° C. in a sealed vessel for 18 hours. An additional amount of triethylsilane (75 μL, 0.47 mmole) and trifluoroacetic acid (36 μL, 0.47 mmole) was added and the mixture was heated at 65° C. in a sealed vessel for an additional 7 hours. The mixture was cooled, quenched with NaHCO₃ and extracted with EtOAc. The combined organic layers were washed with brine, dried (MgSO₄), and concentrated. Purification by chromatography (SiO₂, 95:5:0.3 dichloromethane/methanol/conc. NH₄OH) yielded 34 mg (52%) of the compound of formula 1i wherein R is (E)-(3-quinolinyl)vinyl. MS 936 (M+H)⁺

Step B

The compound from step A (35 mg, 0.037 mmole) was dissolved in methanol (2 ml) and stirred for 17 hours. K₂CO₃ (30 mg, 0.22 mmole) was added and the mixture was stirred for 1 hour. The mixture was diluted with EtOAc, washed with H₂O, washed with brine, dried (MgSO₄), and concentrated. Purification by chromatography (SiO₂, 97:3:0.3 dichloromethane/methanol/conc. NH₄OH) yielded 15 mg (48%) of the title compound as a colorless solid. MS 825 (M+H)⁺

### EXAMPLE 7

### Compound 6 (Formula 1j: R is (E)-[5-(2-bromopyridinyl)]vinyl)

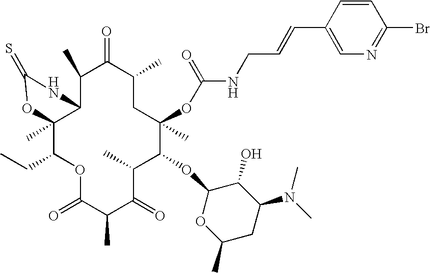 Step A

Compound 2 (50 mg, 0.067 mmole) and (2E)-3-[5-(2-bromopyridinyl)]-2-propenal (43 mg, 0.20 mmole) were treated with triethylsilane (75 μL, 0.47 mmole) and trifluoroacetic acid (36 μL, 0.47 mmole) in acetonitrile (2.0 mls) at 65° C. in a sealed vessel for 18 hours. The mixture was cooled, quenched with NaHCO₃ and extracted with EtOAc. The combined organic layers were washed with brine, dried (MgSO₄), and concentrated. Purification by chromatography (SiO₂, 97:3:0.3 dichloromethane/methanol/conc. NH₄OH) yielded 32 mg (51%) of the compound of formula 1i wherein R is (E)-[5-(2-bromopyridinyl)]vinyl. MS 938 (M+H)⁺

Step B

The compound from step A (32 mg, 0.034 mmole) was dissolved in methanol (2 ml) and stirred for 17 hours. K₂CO₃ (20 mg, 0.14 mmole) was added and the mixture was stirred for 1 hour. The mixture was diluted with EtOAc, washed with H₂O, washed with brine, dried (MgSO₄), and concentrated. Purification by chromatography (SiO₂, 97:3:0.3 dichloromethane/methanol/conc. NH₄OH) yielded 19 mg (66%) of the title compound as a colorless solid. MS 854 (M+H)⁺

### EXAMPLE 8

### Compound 7 (Formula 1i: R is (E)-[5-(2-cyclopropyl)pyrimidinyl]vinyl

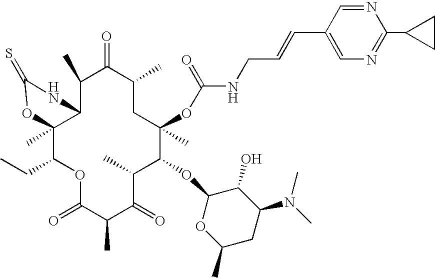 Step A

Compound 2 (50 mg, 0.067 mmole) and (2E)-3-[5-(2-cyclopropyl)pyrimidinyl]-2-propenal (35 mg, 0.20 mmole) were treated with triethylsilane (75 μL, 0.47 mmole) and trifluoroacetic acid (36 μL, 0.47 mmole) in acetonitrile (2.0 mls) at 65° C. in a sealed vessel for 18 hours. The mixture was cooled, quenched with NaHCO₃ and extracted with EtOAc. The combined organic layers were washed with brine, dried (MgSO₄), and concentrated. Purification by chromatography (SiO₂, 97:3:0.3 dichloromethane/methanol/conc. NH₄OH) yielded 31 mg (51%) of the compound of formula 1i wherein R is (E)-[5-(2-cyclopropyl)pyrimidinyl]vinyl. MS 900 (M+H)⁺

Step B

The compound from step A (31 mg, 0.034 mmole) was dissolved in methanol (3 ml) and stirred for 17 hours. K₂CO₃ (25 mg, 0.18 mmole) was added and the mixture was stirred for 1 hour. The mixture was diluted with EtOAc, washed with H₂O, washed with brine, dried (MgSO₄), and concentrated. Purification by chromatography (SiO₂, 97:3:0.3 dichloromethane/methanol/conc. NH₄OH) yielded 21 mg (75%) of the title compound as a colorless solid. MS 816 (M+H)⁺

### EXAMPLE 9

### Compound 8 (Formula 1k: R⁵═CN and R⁶═H)

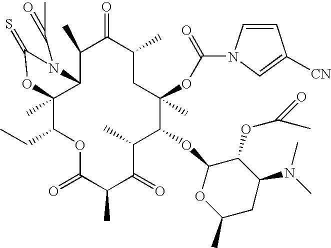

Compound 2 (0.40 g, 0.54 mmole), 2-formyl-4,4-dimethoxybutyronitrile (0.37 g, 2.4 mmol), and trifluoroacetic acid (0.42 ml, 5.4 mmole), in acetonitrile (8 ml) were heated in a sealed vessel at 65° C. for 7 hours. The mixture was cooled, quenched with NaHCO₃ and extracted with EtOAc. The combined organic layers were washed with brine, dried (MgSO₄), and concentrated to give 0.13 g (28%) of the title compound. MS 817 (M+H)⁺

### EXAMPLE 10

### Compound 9 (Formula 1m: R is (2E)-3-(3-quinolinyl)-2-propenyl)

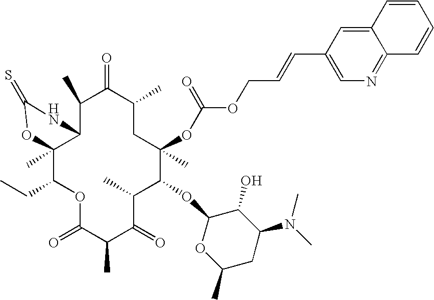 Step A

(2E)-3-(3-quinolinyl)-2-propenol (28 mg, 0.14 mmole) was treated with DBU (23 mg, 0.14 mmole) in THF (2.0 ml) and DMSO (0.1 ml) for 15 minutes. Compound 8 (37 mg, 0.045 mmole) was added and the mixture was stirred for 17 hours. The mixture was diluted with EtOAc, washed with NH₄Cl, washed with brine, dried (MgSO₄), concentrated and chromatographed (SiO₂, 97:3:0.3 dichloromethane/methanol/conc. NH₄OH) to provide the compound of formula 11 wherein R is (2E)-3-(3-quinolinyl)-2-propenyl.

Step B

The compound from step A was treated with methanol (2 ml) for 17 hours. The mixture was concentrated and purified by chromatography (SiO₂, 97:3:0.3 dichloromethane/methanol/conc. NH₄OH) to yield 10 mg (27%) of the title compound. MS 826 (M+H)⁺

### EXAMPLE 11

### Compound 10 (Formula 1m: R is (2E)-3-[5-(2-bromopyridinyl)]-2-propenyl)

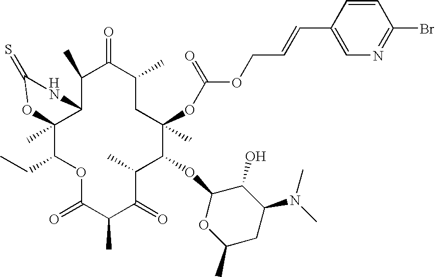 Step A

(2E)-3-[5-(2-bromopyridinyl)]-2-propenol (28 mg, 0.14 mmole) was treated with DBU (25 μL, 0.14 mmole) in THF (2.0 ml) for 15 minutes. Compound 8 (38 mg, 0.047 mmole) was added and the mixture was stirred for 6 hours. The mixture was diluted with EtOAc, washed with NH₄Cl, washed with brine, dried (MgSO4), concentrated and chromatographed (SiO₂, 97:3:0.3 dichloromethane/methanol/conc. NH₄OH) to provide the compound of formula 1l wherein R is (2E)-3-[5-(2-bromopyridinyl)]-2-propenyl.

Step B

The compound from step A was treated with methanol (2 ml) for 17 hours. The mixture was concentrated and purified by chromatography (SiO₂, 97:3:0.3 dichloromethane/methanol/conc. NH₄OH) to yield 4 mg (10%) of the title compound. MS 855 (M+H)⁺

### EXAMPLE 12

### Compound 1j (Formula 1j: R is (Z)-1-fluoro-2-[4-(2-pyrimidinyl)phenyl]-1-vinyl)

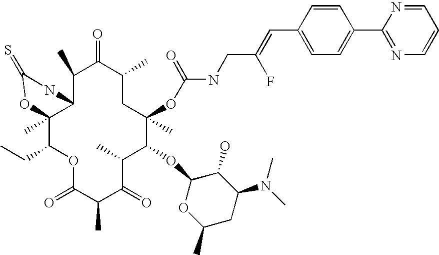 Step A

Compound 2 (35 mg, 0.047 mmole) and (2Z)-2-fluoro-3-[4-(2-pyrimidinyl)phenyl]-2-propenal (32 mg, 0.14 mmole) were treated with triethylsilane (53 μL, 0.33 mmole) and trifluoroacetic acid (25 μL, 0.33 mmole) in acetonitrile (2.0 mls) at 65° C. in a sealed vessel for 18 hours. The mixture was cooled, quenched with NaHCO₃ and extracted with EtOAc. The combined organic layers were washed with brine, dried (MgSO₄), concentrated and chromatographed (SiO₂, 98:2:0.3 dichloromethane/methanol/conc. NH₄OH) to provide the compound of formula 1i wherein R is (Z)-1-fluoro-2-[4-(2-pyrimidinyl)phenyl]-1-vinyl.

Step B

The compound from step A (35 mg, 0.037 mmole) was treated with methanol (2 ml) for 17 hours. K₂CO₃ (20 mg, 0.14 mmole) was added and the mixture was stirred for 2 hours. The mixture was diluted with EtOAc, washed with H₂O, washed with brine, dried (MgSO₄), and concentrated. Purification by chromatography (SiO₂, 95:5:0.3 dichloromethane/methanol/conc. NH₄OH) yielded 6.2 mg (15%) of the title compound as a colorless solid. MS 870 (M+H)⁺

### EXAMPLE 13

### Compound 12 (Formula 1j: R is (Z)-1-fluoro-2-(3-quinolinyl)-1-vinyl

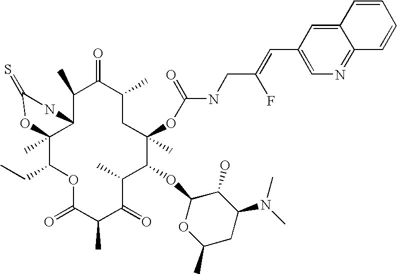 Step A

Compound 2 (35 mg, 0.047 mmole) and (2Z)-2-fluoro-3-(3-quinolinyl)-2-propenal (28 mg, 0.14 mmole) were treated with triethylsilane (53 μL, 0.33 mmole) and trifluoroacetic acid (25 μL, 0.33 mmole) in acetonitrile (2.0 mls) at 65° C. in a sealed vessel for 18 hours. Additional triethylsilane (100 μL, 0.68 mmole) and trifluoroacetic acid (100 μL, 1.32 mmole) were added and the mixture was heated at 65° C. in a sealed vessel for 7 hours. The mixture was cooled, quenched with NaHCO₃ and extracted with EtOAc. The combined organic layers were washed with brine, dried (MgSO₄), concentrated and chromatographed (SiO₂, 98:2:0.3 dichloromethane/methanol/conc. NH₄OH) to provide the compound of formula 1i wherein R is (Z)-1-fluoro-2-(3-quinolinyl)-1-vinyl.

Step B

The compound from step A (35 mg, 0.037 mmole) was treated with methanol (2 ml) for 17 hours. K₂CO₃ (20 mg, 0.14 mmole) was added and the mixture was stirred for 3 hours. The mixture was diluted with EtOAc, washed with H₂O, washed with brine, dried (MgSO₄), and concentrated. Purification by chromatography (SiO₂, 95:5:0.3 dichloromethane/methanol/conc. NH₄OH) yielded 6.2 mg (16%) of the title compound as a colorless solid. MS 843 (M+H)⁺

### EXAMPLE 14

### Compound 13 (Formula 1d R is 2-[4-(2-pyrimidinyl)phenyl]ethyl and E is methyl

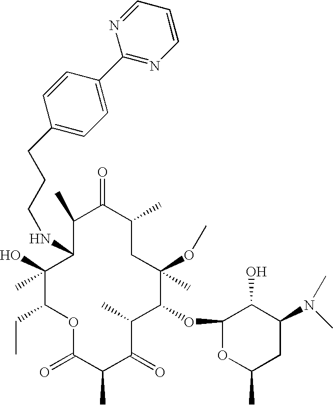 Step A

To a solution of compound XVII, wherein E is methyl, (500 mg, 0.76 mmol) and DMAP ( 93 mg, 0.76 mmol) in THF (10 mL) was added di-tert-butyl dicarbonate (663 mg, 3.04 mmol). The reaction was stirred at room temperature for 24 h before being quenched with H₂O. The mixture was extracted with EtOAc (3×). The combined organic layers were washed with 10% NH₄Cl, sat. aq. NaHCO₃, brine, dried (MgSO₄), and concentrated to give crude compound XVIII, wherein E is methyl.

Step B

Compound XVIII, wherein E is methyl, (crude from step A, 0.76 mmole) was treated with LiOH (0.3 g, 12.5 mmole) in methanol/THF/H₂O (1:1:1, 9 mL) for 24 hours. The mixture was diluted with H₂O and extracted with EtOAc (3×). The combined organic layers were washed with H₂O and brine, dried (MgSO₄), and concentrated. The crude material was then reacted with acetic anhydride (0.29 mL, 3.04 mmol) in the presence of Et₃N (0.63 mL, 4.56 mmol) in CH₂Cl₂ (10 mL) for 16 h. The reaction was quenched with sat. aqueous NaHCO₃ and extracted with EtOAc (3×). The combined organic layers were washed with 10% NH₄Cl, sat. aq. NaHCO₃, brine, dried (MgSO₄), and concentrated. Purification by chromatography (SiO₂, 96:4:0.3 dichloromethane/methanol/conc. NH₄OH) yielded 160 mg (25% for 3 steps) of compound XIX, wherein E is methyl, as a white solid. MS 832 (M+H)⁺

Step C

To a solution of compound XIX, wherein E is methyl, (160 mg, 0.19 mmol) in CH₂Cl₂ (3 mL) at 0° C. was added 2,6-lutidine (0.2 mL, 1.71 mmol) followed by tert-butyldimethylsilyltriflate (0.17 ml, 0.76 mmole). The reaction was warmed to room temperature and stirred for 5 h before being quenched with sat. aqueous NaHCO₃. The mixture was extracted with CH₂Cl₂, and the organic layer was washed with brine, dried (MgSO₄), and concentrated. The resulting residue was treated with KF (0.2 g) in THF/MeOH (4:1, 5 mL) for 15 min. The mixture was diluted with H₂O and extracted with EtOAc. The organic solution was washed with brine, dried (MgSO₄) and concentrated. Purification by chromatography (SiO₂, 95:5:0.3 dichloromethane/methanol/conc. NH₄OH) yielded 80 mg (67%) of compound XX, wherein E is methyl, as a white solid. MS 632 (M+H)⁺

Step D

To a solution of compound XX, wherein E is methyl, (63 mg, 0.1 mmol) and 3-[4-(2-pyrimidinyl)phenyl]propionaldehyde (107 mg, 0.5 mmol) in HOAc (1.5 mL) was added NaCNBH₃ (32 mg, 0.5 mmol). The reaction was heated at 60° C. for 20 min before being cooled to room temperature and quenched with sat. aqueous NaHCO₃. The mixture was extracted with CH₂Cl₂, and the organic layer was washed with brine, dried (MgSO₄) and concentrated. Purification by chromatography (SiO₂, 96:4:0.3 dichloromethane/methanol/conc. NH₄OH) yielded 30 mg (36%) of a white solid (compound XXII, wherein R=2-[4-(2-pyrimidinyl)phenyl]ethyl) and E is methyl. MS 828 (M+H)⁺

Step E

The compound from step D (30 mg, 0.036 mmol) was reacted with Dess-Martin reagent (38 mg, 0.09 mmol) in CH₂Cl₂ (2 mL) for 15 min. The reaction was quenched with H₂O and extracted with CH₂Cl₂. The organic layer was washed with brine, dried (MgSO₄) and concentrated. The resulting residue was then stirred in MeOH (5 mL) at room temperature for 16 h. The solution was concentrated and the residue purified by chromatography (SiO₂, 95:5:0.3 dichloromethane/methanol/conc. NH₄OH) to yield 6 mg (21% for 2 steps) of the title compound as a white solid. MS 784 (M+H)⁺

### REFERENCE EXAMPLE 1

### (2E)-3-[4-(2-pyrazinyl)phenyl]-2-propenal

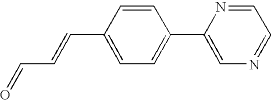 Step A

Ethanol (13 ml) and 1.0M Na₂CO₃ (27.5 ml) were added to a suspension of 2-chloropyrazine (4.0 g, 34.6 mmole), 4-formylphenylboronic acid (6.8 g, 45.0 mmole), and tetrakis(triphenylphosphine)palladium(0) (2.0 g, 1.7 mmole) in toluene (55 ml). The mixture was refluxed for 18 hours then cooled, diluted with EtOAc, washed with NaHCO₃, washed with brine, dried (MgSO₄) and concentrated. Purification by chromatography (SiO₂, 4:1 hexanes/EtOAc) yielded 6.2 g (97%) of 4-(2-pyrazinyl)benzaldehyde.

Step B

The compound from step A (6.2 g, 33.5 mmole), (1,3-dioxolan-2-yl-methyl)triphenylphosphonium bromide (22.6 g, 52.7 mmole), tris[2-(2-methoxyethoxy)ethyl]amine (11.2 ml, 34.9 mmole), sat. aq. K₂CO₃ (150 mls), and CH₂Cl₂ (150 ml) were heated at reflux for 17 hours. The mixture was cooled and the aqueous layer was washed with CH₂Cl₂ (2×). The combined organic layers were washed with NaHCO₃, brine, dried (MgSO₄), and concentrated.

Step C

The crude product from step B was treated with 10% HCl (aq.) (80 ml) in THF (80 ml) for 1 hour. Most of the THF was removed in vacuo and the mixture was cooled to 0° C. and basified (pH>10) with 10% NaOH, then extracted with EtOAc (3×). The combined organic layers were washed with H₂O, washed with brine, dried and concentrated. Purification by chromatography (SiO₂, 2:1 hexanes/EtOAc) yielded 6.2 g (88%) of the title compound.

### REFERENCE EXAMPLE 2

### (2E)-3-[4-(3-pyridazinyl)phenyl]-2-propenal

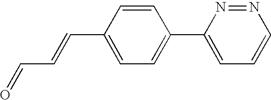 Step A

3-(2H)-pyridazinone (5.0 g, 52.0 mmole) was treated with phosphorous oxychloride (17 ml, 179 mmole) at 85° C. for 4.5 hours. The mixture was poured into 400 g ice/H₂O, basified (pH>10) with 50% NaOH, and extracted with EtOAc (4×). The combined organic layers were washed with brine, dried (MgSO₄), and concentrated. The material was run through a Hak-Pak (SiO₂, 1:1 hexanes/EtOAc) to give 2.8 g (46%) of 3-chloropyridazinone.

Step B

Ethanol (10 ml) and 1.0M Na₂CO₃ (18 ml) were added to a suspension of the compound from step A (2.8 g, 24.0 mmole), 4-formylphenylboronic acid (4.7 g, 31.2 mmole), and tetrakis(triphenylphosphine)palladium(0) (1.4 g, 1.2 mmole0 in toluene (35 ml). The mixture was refluxed for 20 hours then cooled, diluted with EtOAc, washed with NaHCO₃, washed with brine, dried (MgSO₄) and concentrated. Purification by chromatography (SiO₂, 4:1 hexanes/EtOAc) yielded 4.1 g (93%) of 4-(3-pyridazinyl)benzaldehyde.

Step C

The compound from step B (4.1 g, 22.3 mmole), (1,3-dioxolan-2-yl-methyl)triphenylphosphonium bromide (15.0 g, 35.0 mmole), tris[2-(2-methoxyethoxy)ethyl]amine (7.4 ml, 23.2 mmole), sat. aq. K₂CO₃ (120 mls), and CH₂Cl₂ (120 ml) were heated at reflux for 17 hours. The mixture was cooled and the aqueous layer was washed with CH₂Cl₂ (2×). The combined organic layers were washed with NaHCO₃, washed with brine, dried (MgSO₄), and concentrated.

Step D

The compound from step C was treated with 10% HCl (aq.) (60 ml) in THF (60 ml) for 1 hour. Most of the THF was removed in vacuo and the mixture was cooled to 0° C. and basified (pH>10) with 10% NaOH, then extracted with EtOAc (3×). The combined organic layers were washed with H₂O, washed with brine, dried (MgSO₄) and concentrated. Purification by chromatography (SiO₂, 2:1 hexanes/EtOAc) yielded 3.9 g (83%) of the title compound.

### REFERENCE EXAMPLE 3

### (2E)-3-(3-quinolinyl)-2-propenal

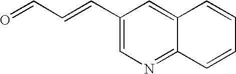

A mixture of 3-quinolinecarboxaldehyde (2.0 g, 12.7 mmole), (1,3-dioxolan-2-yl-methyl)triphenylphosphonium bromide (8.6 g, 20.0 mmole), tris[2-(2-methoxyethoxy)ethyl]amine (4.2 ml, 4.3 mmole), sat. aq. K₂CO₃ (50 mls), and CH₂Cl₂ (50 ml) were heated at reflux for 5 hours. The aqueous layer was extracted with CH₂Cl₂ (2×) and the combined organic layers were washed with brine, dried (MgSO₄), and concentrated. The crude material was treated with 10% HCl (aq.) (60 ml) in THF (60 ml) for 1 hour. Most of the THF was removed in vacuo and the mixture was cooled to 0° C. and basified (pH>10) with 10% NaOH, then extracted with EtOAc (3×). The combined organic layers were washed with H₂O, washed with brine, dried (MgSO₄) and concentrated. Purification by chromatography (SiO₂, 1:1 hexanes/EtOAc) yielded 1.8 g (77%) of the title compound. MS 184 (M+H)⁺

### REFERENCE EXAMPLE 4

### (2E)-3-[5-(2-bromopyridinyl)]-2-propenal

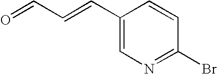

Isopropyl magnesium bromide (2.0 N, 4.55 ml, 9.1 mmole) was added to a solution of 2,5-dibromopyridine (2.2 g, 9.1 mmole) in THF (5.0 ml). The mixture was stirred for 1 hour then 3-dimethylaminoacrolein (1.1 g, 11.34 mmole) was added. The mixture was warmed to ambient temperature and stirred for 2 hours. The mixture was treated HCl (3.0 N, 10 ml) for 10 minutes then basified (pH>10) with 50% NaOH. The mixture was extracted with EtOAc (3×). The combined organic layers were washed with brine, dried (MgSO₄), and concentrated. Purification by chromatography (SiO₂, 4:1 hexanes/EtOAc) yielded 2.1 g (quantitative) of the title compound. MS 214 (M+H)⁺

### REFERENCE EXAMPLE 5

### (2E)-3-(3-quinolinyl)-2-propenol

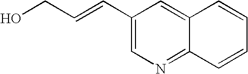

(2E)-3-(3-quinolinyl)-2-propenal (0.5 g, 2.7 mmole) was treated with sodium borohydride (0.31 g, 8.2 mmole) in THF (5 ml) at 0° C. for 30 minutes. The mixture was quenched with H₂O and extracted with EtOAc (3×). The combined organic layers were washed with brine, dried (MgSO₄), and concentrated. Purification by chromatography (SiO₂, 1:1 hexanes/EtOAc) yielded 0.38 g (75%) of the title compound. MS 186 (M+H)⁺

### REFERENCE EXAMPLE 6

### (2E)-3-[5-(2-bromopyridinyl)]-2-propenol

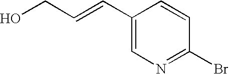

The compound from Reference Example 4 (0.34 g, 1.6 mmole) was treated with sodium borohydride (0.24 g, 6.4 mmole) at 0° C. in THF (20 ml) for 30 minutes. The mixture was quenched with H₂O, the majority of the THF was removed in vacuo, and the mixture was extracted with EtOAc (3×). The combined organic layers were washed with brine, dried (MgSO₄), and concentrated. Purification by chromatography (SiO₂, 3:1 hexanes/EtOAc) yielded 0.28 g (97%) of the title compound. MS 185 (M+H)⁺

### REFERENCE EXAMPLE 7

### (2Z)-2-fluoro-3-[4-(2-pyrimidinyl)phenyl]-2-propenal

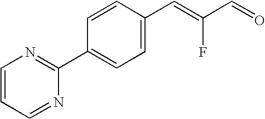 Step A:

Triethyl 2-fluoro-2-phosphonoacetate (1.55 mL, 7.64 mmol) was added to a suspension of MgBr₂ (1.68 g, 9.12 mmol) in THF (20 mL). The resulting mixture was cooled to 0° C., triethylamine (1.20 mL, 8.61 mmol) was added, and stirring was continued for 1 h at 0° C. A solution of 4-(2-pyrimidinyl)benzaldehyde (1.00 g, 5.43 mmol, prepared as described in WO 9828264) in THF (10 mL) was added via cannula and an additional amount of THF (5 mL) was used to rinse the transfer flask and cannula. The resulting mixture was stirred for 3 h at 0° C., quenched with 10% aq. ammonium chloride (5 mL), and concentrated to a small volume. The concentrate was diluted with ethyl acetate (50 mL), washed with 10% aq. ammonium chloride, sat. aq. NaHCO₃, and brine (50 mL each), dried (MgSO₄), and concentrated. Purification by chromatography (SiO₂, 3:1 hexane/ethyl acetate) provided 1.27 g of the title compound as a 3:1 mixture with its E isomer. Recrystallization from 2-propanol provided 0.76 g (51%) of ethyl 2-fluoro-3-[4-(2-pyrimidinyl)phenyl]-2-propenoate containing ca. 1% of the E isomer. MS 273 (M+H)⁺.

Step B:

Diisobutylaluminum hydride (1.0 M solution in THF, 5.5 mL, 5.50 mmol) was added dropwise to a 0° C. solution of the product from step A (500 mg, 1.84 mmol) in methylene chloride (15 mL). The resulting solution was stirred for 10 min at 0° C., quenched with methanol (0.25 mL) followed by 15% aq. Rochelle salt (20 mL), and allowed to stir at room temperature for 4 h. The layers were separated and the aqueous layer was extracted with methylene chloride (20 mL). The combined organic layers were dried (MgSO₄) and concentrated to provide 415 mg (98%) of 2-fluoro-3-[4-(2-pyrimidinyl)phenyl]-2-propen-1-ol as a colorless solid. MS 231 (M+H)⁺.

Step C:

The compound from step B (2.0 g, 8.7 mmole) was treated with Dess-Martin reagent (3.9 g, 9.1 mmole) in CH₂Cl₂ for 4 hours. The mixture was quenched with NaHCO₃ and extracted with CH₂Cl₂ (3×). The combined organic layers were washed with brine, dried (MgSO₄), and concentrated. Purification by chromatography (SiO₂, 2:1 hexanes/EtOAc) yielded 1.7 g (84%) of the title compound. MS 229 (M+H)⁺

### REFERENCE EXAMPLE 8

### (2Z)-2-fluoro-3-(3-quinolinyl)-2-propenal

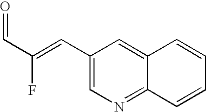 Step A

2-Fluoro-3-quinolin-3-ylacrylic acid ethyl ester (1.0 g, 4.1 mmole; prepared in a similar manner as for Reference Example 7, but with 3-quinolinecarboxaldehyde in place of 4-(2-pyrimidinyl)benzaldehyde) was treated with DIBAL (1.0 M in hexanes, 4.9 ml, 4.9 mmole) at −78° C. for 30 minutes. The mixture was quenched with HCl (1.0 M, 25 ml) and extracted with EtOAc (3×). The combined organic layers were washed with brine, dried (MgSO₄), and concentrated.

Step B

The material from step A was treated with Dess-Martin reagent (1.8 g, 4.2 mmole) in CH₂Cl₂ for 5 hours. The mixture was quenched with NaHCO₃ and extracted with CH₂Cl₂. The combined organic layers were washed with brine, dried (MgSO4), and concentrated. Purification by chromatography (SiO₂, 2:1 hexanes/EtOAc) yielded 0.58 g (71%) of the title compound. MS 202 (M+H)⁺

### REFERENCE EXAMPLE 9

### (2E)-3-(2-cyclopropylpyrimidin-5-yl)propenal

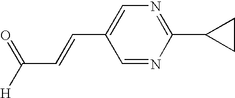 Step A:

To a solution of ethyl 3-(2-cyclopropylpyrimidin-5-yl)acrylate (prepared as described in WO 00/66566) (0.77 g, 3.5 mmol) in CH₂Cl₂ (35 mL) at −78 ° C. was added diisobutylaluminum hydride (1.0 M solution in hexane, 9 mL). The reaction was stirred at −78° C. for 30 min before being quenched with MeOH (5 mL) followed by 2.5 N aq. NaOH (20 mL). The mixture was extracted with CH₂Cl₂ (50 mL×4), and the resulting organic solution was washed with brine, dried (MgSO₄) and concentrated.

Step B:

A solution of the crude alcohol from step A and MnO₂ (2 g) in CH₂Cl₂ (20 mL) was stirred at room temperature for 16 h. Filtration, concentration and purification by chromatography (silica gel, 98:2 dichloromethane/methanol) gave 0.32 g (52%) of the title compound as a yellow solid MS 175 (M+H)⁺.

1. A compound of Formula 1: 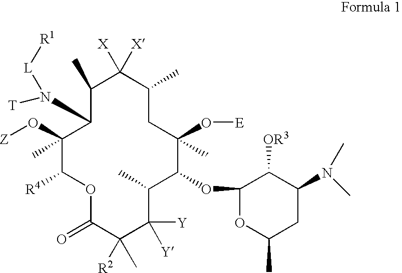
wherein
R¹ is selected from the group consisting of hydrogen, optionally substituted alkyl, optionally substituted alkenyl, optionally substituted alkynyl, cycloalkyl, optionally substituted aryl, and optionally substituted heteroaryl;
R² is selected from the group consisting of hydrogen, halogen, and hydroxy;
R³ is hydrogen or a hydroxy protecting group;
R⁴ is selected from the group consisting of hydrogen, C₁-C₁₀-alkyl, C₂-C₁₀-alkenyl, C₂-C₁₀-alkynyl, aryl, heteroaryl, heterocyclo, aryl(C₁-C₁₀)alkyl, aryl(C₂-C₁₀)alkenyl, aryl(C₂-C₁₀)alkynyl, heterocyclo(C₁-C₁₀)alkyl, heterocyclo(C₂-C₁₀)alkenyl, and heterocyclo(C₂-C₁₀)alkynyl, C₃-C₆-cycloalkyl, C₅-C₈-cycloalkenyl, alkoxyalkyl containing 1-6 carbon atoms in each alkyl or alkoxy group, and alkylthioalkyl containing 1-6 carbon atoms in each alkyl or thioalkyl group;
L is absent or C(O);
T is hydrogen;
Z is hydrogen, or T and Z may be taken together to form a thiocarbonyl group;
E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein
Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 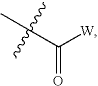
wherein
W is selected from the group consisting of
(1) a substituted pyrrole of the formula 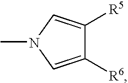
wherein
R⁵ and R⁶ are independently selected from the group consisting of hydrogen, CN, nitro, —C(O)R⁷, —C(O)OR⁷, —C(O)NR⁷R⁸, —SO₂R⁷, C₁-C₈-alkyl, C₂-C₈-alkenyl, C₂-C₈-alkynyl, C₃-C₈-cycloalkyl, C₅-C₈-cycloalkenyl, aryl, and heteroaryl, wherein
R⁷ and R⁸ are independently selected from the group consisting of hydrogen, alkyl, alkenyl, alkynyl, cycloalkyl, aryl, heteroaryl, heterocyclo, aralkyl, heteroaralkyl, and heterocycloalkyl;
(2) NHR⁹, wherein
R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl; and
(3) OR⁹, wherein
R⁹ is independently selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl;
X and X′, together with the carbon atom to which they are attached, form C═O, C═NR_{c}, or C═NOR_{c}, wherein R_{c} is independently selected from the group consisting of hydrogen, alkyl, alkenyl and alkynyl; and
Y and Y′, together with the carbon atom to which they are attached, form C═O, —CHOH, C═NR_{c}, or C═NOR_{c}, wherein R_{c} is independently selected from the group consisting of hydrogen, alkyl, alkenyl and alkynyl;
with the following provisos:
1) when L is absent and T and Z combine to form a thiocarbonyl group, R¹ is hydrogen;
2) when E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, T and Z are both hydrogen;
3) when T and Z are both hydrogen, E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein
Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 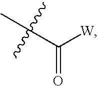
wherein
W is NHR⁹, wherein
R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl;
or an optical isomer, enantiomer, diastereomer, racemate or racemic mixture thereof, or a pharmaceutically acceptable salt, esters or pro-drugs thereof.

2. A pharmaceutical composition comprising a compound of claim 1 and a pharmaceutically acceptable carrier.

3. A method of treating a subject having a condition caused by or contributed to by bacterial infection, which comprises administering to the subject a therapeutically effective amount of the compound of Formula 1 as defined in claim 1.

4. A method of preventing a subject from suffering from a condition caused by or contributed to by bacterial infection, which comprises administering to the subject a prophylactically effective amount of the compound of Formula 1 as defined in claim 1.

5. The method of claim 3 or 4 wherein the condition is selected from community-acquired pneumonia, upper and lower respiratory tract infections, skin and soft tissue infections, meningitis, hospital-acquired lung infections, and bone and joint infections.

6. The method of claim 3 or 4 wherein the bacterium is selected from S. aureus, S. epidermidis, S. pneumoniae, Enterococcus spp., Moraxella catarrhalis and H. influenzae.

7. The method of claim 3 or 4 wherein the bacterium is a Gram-positive coccus.

8. The method of claim 3 wherein the Gram-positive coccus is antibiotic-resistant.

9. The method of claim 8 wherein the Gram-positive coccus is erythromycin-resistant.

10. The method of claim 3 wherein the bacterium is a Gram-positive or Gram-negative respiratory pathogen.

11. A process for preparation of a compound having the formula, 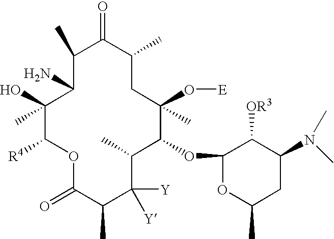
wherein
R³ and R⁴, are as previously defined, Y and Y′, together with the carbon atom to which they are attached, are as previously defined, E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 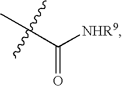
wherein W is NHR⁹, wherein R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl,
comprising:
a) treating a compound having the formula 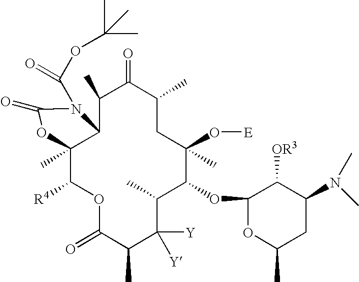
wherein
R³ and R⁴ are as previously defined, and Y and Y′, together with the carbon atom to which they are attached, are as previously defined and in addition are CHOCO₂C(CH₃)₃, E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 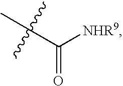
wherein W is NHR⁹, wherein R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl,
with a suitable inorganic base in a suitable solvent;
b) optionally reprotecting the 2′-hydroxy group;
c) treating the product of step (a) or step (b) with a silylating agent in the presence of a pyridine base;
c) treating the product of step (c) with a fluoride salt; and
d) when R³ is a hydroxy protecting group, optionally deprotecting the 2′-hydroxy group.

12. A process for preparation of a compound having the formula, 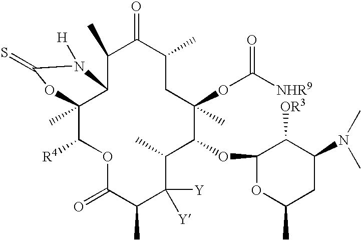
wherein
R³ and R⁴, are as previously defined, Y and Y′, together with the carbon atom to which they are attached, are as previously defined and
R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl,
comprising:
a) treating a compound having the formula 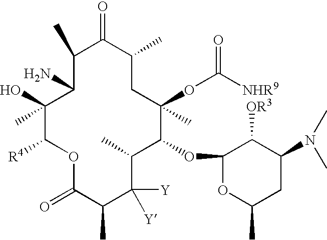
wherein
R³ and R⁴ are as previously defined, and Y and Y′, together with the carbon atom to which they are attached, are as previously defined, and
R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl,
with carbon disulfide (CS₂) in the presence of a suitable tertiary amine base in a suitable solvent,
b) when R³ is a hydroxy protecting group, optionally deprotecting the 2′-hydroxy group.

13. A process for preparation of a compound having the formula, 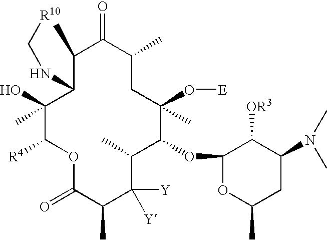
wherein
R³ and R⁴, are as previously defined, Y and Y′, together with the carbon atom to which they are attached, are as previously defined, R¹⁰ is is selected from the group consisting of hydrogen, optionally substituted alkyl, optionally substituted alkenyl, optionally substituted alkynyl, cycloalkyl, optionally substituted aryl, and optionally substituted heteroaryl; E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 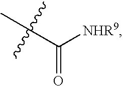
wherein R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO2Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl,
comprising:
a) treating a compound having the formula 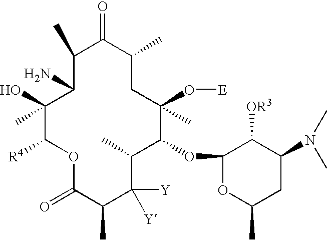
wherein
R³ and R⁴ are as previously defined, and Y and Y′, together with the carbon atom to which they are attached, are as previously defined, E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO2Rₐ, —NRₐR_{b}, —CONRₐR_{b}, —OCONRₐR_{b}, —NHCORₐ, —NHCOORₐ, —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 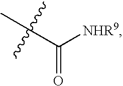
wherein R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl,
with an aldehyde R¹⁰CHO, wherein R¹⁰ is as defined above, in the presence of a suitable reducing agent and an acid catalyst in a suitable solvent,
b) when R³ is a hydroxy protecting group, optionally deprotecting the 2′-hydroxy group.

14. A compound of claim 1 having the formula, 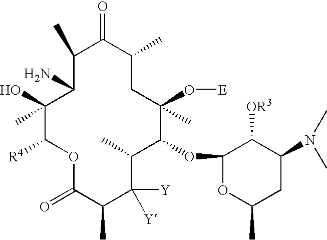
wherein,
R³ and R⁴ are as previously defined, and Y and Y′, together with the carbon atom to which they are attached, are as previously defined, E is selected from the group consisting of optionally substituted C₃-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl, and 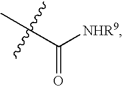
wherein R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl.

15. A compound of claim 1 having the formula, 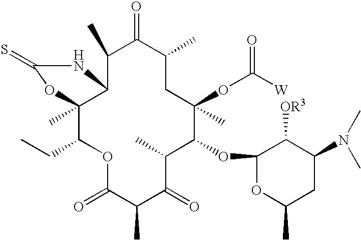
wherein,
R³ is as previously defined; and
W is selected from the group consisting of
(1) NHR⁹, wherein
R⁹ is independently selected from the group consisting of hydrogen, optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO2Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl; and
(2) OR⁹, wherein
R⁹ is independently selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, and —NHCONRₐR_{b}, wherein Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl.

16. A compound of claim 1 having the formula, 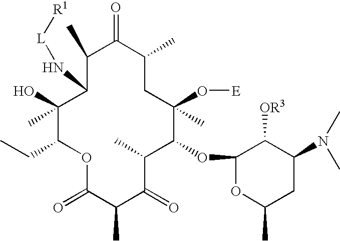
wherein,
R¹ is selected from the group consisting of hydrogen, optionally substituted alkyl, optionally substituted alkenyl, optionally substituted alkynyl, cycloalkyl, optionally substituted aryl, and optionally substituted heteroaryl;
R³ is as previously defined;
L is absent; and
E is selected from the group consisting of optionally substituted C₁-C₈-alkyl, optionally substituted C₃-C₈-alkenyl, and optionally substituted C₃-C₈-alkynyl, wherein the substituents are independently selected from halogen, alkyl, alkenyl, alkynyl, cycloalkyl, oxo, aryl, heteroaryl, heterocyclo, CN, nitro, —COORₐ, —OCORₐ, —ORₐ, —SRₐ, —SORₐ, —SO₂Rₐ, —NRₐR_{b}, —CONRₐR_{b}, —OCONRₐR_{b}, —NHCORₐ, —NHCOORₐ, —NHCONRₐR_{b}, wherein
Rₐ and R_{b} are independently selected from hydrogen, alkyl, alkenyl, alkynyl, cycloalkyl, aryl, heteroaryl, heterocyclo, aralkyl, heteroaralkyl, and heterocycloalkyl.

17. A compound of claim 1 having the formula, 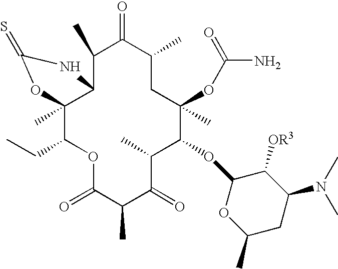
wherein R³ is as previously defined.

18. A compound of claim 1 having the formula, 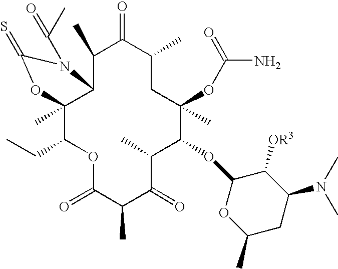
wherein R³ is as previously defined.

19. A compound of claim 1 having the formula,

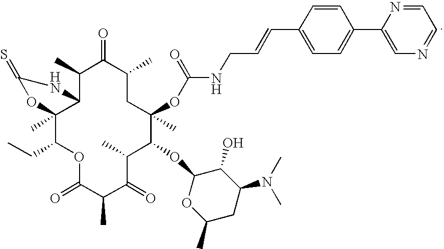

20. A compound of claim 1 having the formula,

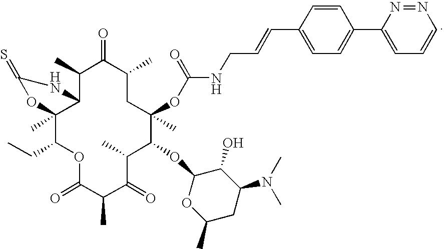

21. A compound of claim 1 having the formula,

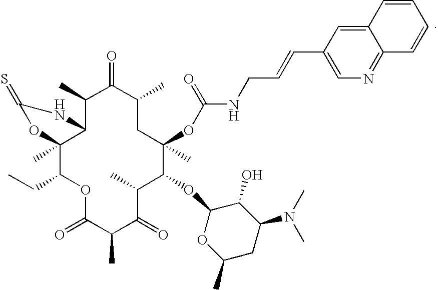

22. A compound of claim 1 having the formula,

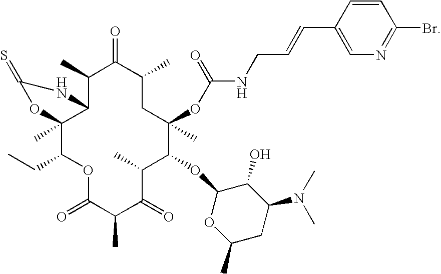

23. A compound of claim 1 having the formula,

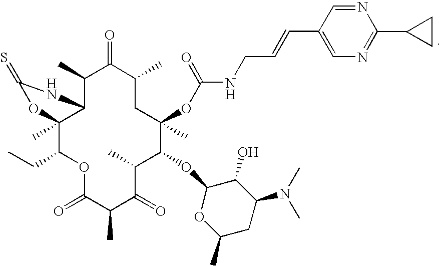

24. A compound of claim 1 having the formula,

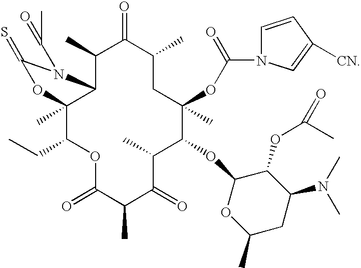

25. A compound of claim 1 having the formula,

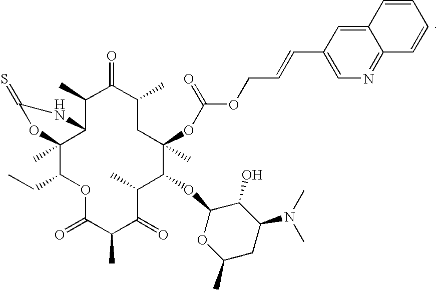

26. A compound of claim 1 having the formula,

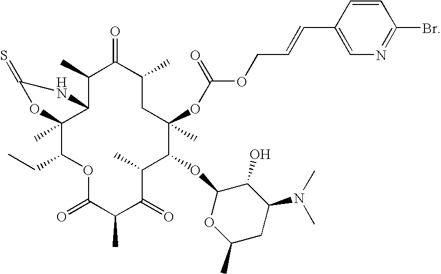

27. A compound of claim 1 having the formula,

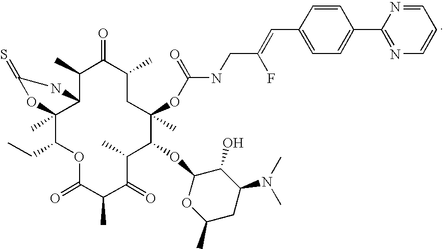

28. A compound of claim 1 having the formula,

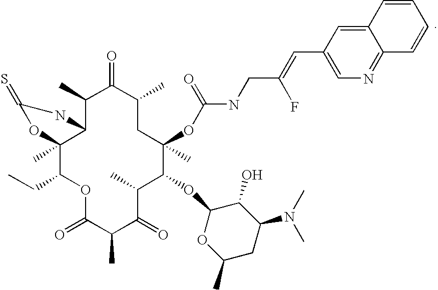

29. A compound of claim 1 having the formula, [chemical structure]
